(19) United States
(12) Patent Application Publication
(10) Pub. No.: US 2016/0120963 A1
(43) Pub. Date: May 5, 2016

(54) BILE SALT HYDROLASE BSH1 FOR REGULATING WEIGHT GAIN, SERUM CHOLESTEROL LEVELS, AND LIVER TRIGLYCERIDES IN A MAMMAL; BACTERIA STRAINS EXPRESSING BSH1 VARIANTS

(71) Applicant: UNIVERSITY COLLEGE CORK NATIONAL UNIVERSITY OF IRELAND, CORK, Co. Cork (IE)

(72) Inventors: Susan Joyce, Co.Cork (IE); Cormac Gahan, Co. Cork (IE); Colin Hill, Co. Cork (IE)

(73) Assignee: University College Cork, National University of Ireland, Co. Cork (IE)

(21) Appl. No.: 14/897,484

(22) PCT Filed: Jun. 12, 2014
(86) PCT No.: PCT/EP2014/062294

§ 371 Date: Dec. 10, 2015

(30) Foreign Application Priority Data
Jun. 12, 2013 (EP) 13171762.1

(51) Int. Cl. A61K 38/50; A61K 9/00; A61K 35/747

(52) U.S. Cl. CPC A61K 38/50; A61K 35/747; A61K 9/0053; A23K 1/009; A23L 1/3014; C12Y 305/01024; A23V 2002/00

(57) ABSTRACT

A non-therapeutic method of reducing weight gain, serum cholesterol levels, or liver triglyceride levels, in a non-obese mammal, comprises the step of administering to the gut of a mammal an effective amount of a bacteria expressing a BSH1 enzyme of SEQUENCE ID NO: 1, or a functional variant thereof; a BSH1 enzyme of SEQUENCE ID NO: 1, or a functional variant thereof for use as a medicament; isolated bacterial strains expressing functional variants of BSH1.

### TECHNICAL FIELD

The invention relates to methods of regulating weight gain, serum cholesterol levels, and liver triglycerides in a mammal. In particular, the invention relates to a method of treatment of a disease or condition in a mammal that is associated with weight gain, serum cholesterol levels, and/or liver triglycerides in a non-obese mammal, for example obesity or hypercholesteremia.

### BACKGROUND TO THE INVENTION

The gastrointestinal microbiota exerts a major influence on host energy metabolism and adiposity however the precise microbial activities that influence lipid metabolism in the host remain largely unexplored. Large scale sequencing studies have catalogued the genetic composition of the human gut microbiota (the microbiome), aiding our understanding of core microbial genes whose products are predicted to influence host metabolism. However studies elucidating the influence of individual bacterial gene sets on systemic metabolic processes in the host are lacking There is currently a significant need for functional categorization of both gut-specific and gut-enriched microbial activities in order to determine the relevance of specific gene sets in a physiological or pathological context.

Bile acids are the main functional components of bile secretions that play a role in the emulsification of dietary lipids and also act as signalling molecules in the host, triggering cellular farnesoid X receptor (FXR)- and G-protein coupled receptor (TGR5)-mediated host responses. Bile acids influence the composition of the gastrointestinal microbiota and in turn are chemically modified by bacterial enzymes in the gut. Many consider bile acids as mediators of a reciprocal microbe-host crosstalk with the ability to influence host metabolic pathways and the potential to influence microbial community structure. Bile acids are synthesized in hepatocytes as cholesterol moieties conjugated to either a taurine or a glycine amino acid and are stored in the gallbladder prior to secretion into the duodenum via the common bile duct. Bacterial enzymes in the gut significantly modify bile acids, a process which in turn influences host bile acid synthesis through a feedback mechanism in which the hepatic enzymes involved in bile acid synthesis (including Cyp7A1 and Cyp27A1) are regulated.

In particular, bacterial bile salt hydrolase (BSH) enzymes in the gut catalyse an essential gateway reaction in the metabolism of bile acids. BSH enzymes cleave the amino acid side-chain of glyco- or tauro-conjugated bile acids to generate unconjugated bile acids (cholic and chenodeoxycholic acids), which are then amenable to further bacterial modification to yield secondary bile acids (deoxycholic and lithocholic acid). It has previously been shown that functional BSH activity is a conserved microbial adaptation that is unique to the gut associated microbiota and is distributed across the major bacterial divisions, as well as archaeal species in the GI tract. It has previously been demonstrated that BSH contributes to bile tolerance in gut bacteria and hypothesized that the evolution of BSH activity is governed by host-driven selection.

### STATEMENTS OF INVENTION

The Applicant has discovered that expression of certain cloned bacterial BSH enzymes in the mammalian GI tract significantly modifies plasma bile acid profiles in gnotobiotic mice and influences both local and systemic gene expression profiles in pathways governing lipid metabolism, metabolic signalling events, circadian rhythm and immune function (FIGS. 1-4). Specifically, the Applicant shows that elevating the activity of specific BSH enzymes in conventionally raised mice can significantly reduce weight gain, serum cholesterol and liver tryglycerides in these animals (FIG. 5). The BSH enzymes typically have at least 90% sequence identity, and ideally at least 96% sequence identity, with the BSH1 enzyme of Lactobacillus salivarius JCM1046 (SEQUENCE ID NO: 1)—examples of suitable bacteria derived from pigs and humans are provided in Tables 1-3. FIG. 12 shows the bile acid deconjugation effects of three strains of bacteria expressing BSH1 enzymes having at least 90% sequence identity with SEQUENCE ID NO: 1.

Thus, in a preferred aspect, the invention provides a non-therapeutic method of reducing weight gain, serum cholesterol levels, or liver triglyceride levels, in a non-obese mammal, comprising the step of administering to the gut of a mammal an active agent comprising a bacteria that expresses BSH1 enzyme of SEQUENCE ID NO: 1, or a functional variant thereof having at least 90% sequence identity with SEQUENCE ID NO: 1. Examples of bacteria that expresses BSH1 enzymes having at least 90% sequence identity with SEQUENCE ID NO: 1 are provided in Table 1 below.

In a another aspect, the invention relates to a method of reducing one, more or all of weight gain, serum cholesterol levels, and liver triglyceride levels, or modulating circadian rhythms, in a mammal, comprising the step of administering to the gut of a mammal an effective amount of a BSH1 enzyme of SEQUENCE ID NO: 1, or a functional variant thereof (hereafter “active of the invention”).

The active of the invention may be administered in the form of an enzyme, typically in a suitable formulation, for example a liposome or microcapsule formulation designed to release the active in the gut of the mammal. Such liposome or microcapsule formulations will be known to those skilled in the art, and are described in more detail below.

In another aspect, the invention relates to a method of reducing one, more or all of weight gain, serum cholesterol levels, and liver triglyceride levels, or regulating circadian rhythms, in a mammal, comprising the step of administering to the mammal an effective amount of bacteria, preferably a probiotic bacteria, that expresses a BSH1 enzyme of SEQUENCE ID NO: 1, or a functional variant thereof.

Thus, in an alternative embodiment, the active may be administered by administration to the gut of the mammal of a bacteria that expresses the active of the invention. The bacteria may be a bacteria that naturally expresses the active of the invention—an example of such a bacteria is Lactobacillus salivarius JCM1046 (Korean Collection of Type Cultures, KCTC 3156 http://www.straininfo.net/strains/171296) Alternatively, the bacteria may be genetically modified to express, ideally stably express, the active of the invention—an example of such a bacteria is the commensal Escherichia coli strain MG1655, which is genetically modified to express the BSH1 gene of SEQUENCE ID NO: 1. Typically, the bacteria is genetically modified using the mini-Tn7 transposon system. Suitably, the gene encoding the active of the invention is integrated into the host genome downstream of the glmS gene.

Preferably the bacteria is a bacteria that exhibits elevated expression of the active of the agent.

Suitably, the bacteria is a probiotic bacteria. Preferably, the bacteria is selected from the group consisting of APC1484 to APC1502.

In a third aspect, the invention relates to a bacteria, preferably a probiotic bacteria, that is genetically engineered to express a BSH1 enzyme of SEQUENCE ID NO: 1, or a functional variant thereof.

The invention also provides a recombinant vector comprising a nucleic acid encoding a BSH1 enzyme of SEQUENCE ID NO: 1, or a functional variant thereof, optionally under the control of a constitutive promotor. Details of constitutive promotors will be well known to those skilled in the art.

The invention also relates to a host cell transformed by a recombinant vector of the invention (hereafter “host cell of the invention”).

The invention also relates to a BSH1 enzyme of SEQUENCE ID NO: 1, or a functional variant thereof, for use as a medicament.

The invention also relates to a BSH1 enzyme of SEQUENCE ID NO: 1, or a functional variant thereof, for use as an antibacterial agent or an antibiotic.

The invention also relates to a BSH1 enzyme of SEQUENCE ID NO: 1, or a functional variant thereof, for use in treating or preventing a disease or condition characterised by weight gain, elevated cholesterol levels, elevated liver triglyceride levels. Examples of such diseases include obesity, hypercholesterolemia, cardiovascular disease and metabolic disease.

The invention also relates to a BSH1 enzyme of SEQUENCE ID NO: 1, or a functional variant thereof, for use in treating or preventing a disease or condition characterised by disregulated circadian rhythm, for example sleep apnoea.

The invention also relates to a bacteria that expresses BSH1 enzyme of SEQUENCE ID NO: 1, or a functional variant thereof, for use in treating or preventing a disease or condition characterised by disregulated circadian rhythm, for example sleep apnoea. The bacteria may be genetically modified to express the active of the invention. Preferably, the bacteria is a probiotic bacteria. Ideally, the bacteria exhibits elevated expression of the active of the invention.

The invention also relates to a pharmaceutical composition comprising BSH1 enzyme of SEQUENCE ID NO: 1, or a functional variant thereof, in combination with a suitable pharmaceutical excipient.

The invention also relates to a pharmaceutical composition comprising a bacteria that expresses, ideally exhibits elevated expression, of a BSH1 enzyme of SEQUENCE ID NO: 1, or a functional variant thereof, in combination with a suitable pharmaceutical excipient. Preferably the bacteria is a probiotic bacteria.

The invention also relates to a formulation comprising a BSH1 enzyme of SEQUENCE ID NO: 1, or a functional variant thereof, or a bacteria that expresses, ideally exhibits elevated expression, of a BSH1 enzyme of SEQUENCE ID NO: 1, or a functional variant thereof. Suitably, the formulation is a pharmaceutical formulation and additionally comprises a pharmaceutically acceptable carrier. Alternatively, the formulation may be a comestible product, for example a food product. Ideally, the food product is a fermented food, for example a fermented dairy product such as a yoghurt. The formulation may also be a hygiene product, for example an antibacterial formulation, or a fermentation product such as a fermentation broth. For formulations that comprise the BSH1 enzyme or variant thereof, it will be appreciated that the enzyme may be directly added to the formulation, or it may be produced in-situ in the formulation by a bacteria.

The invention also relates to BSH1 enzyme of SEQUENCE ID NO: 1, or a functional variant thereof, for use in treating or preventing a metabolic disease or metabolic syndrome.

The invention also relates to BSH1 enzyme of SEQUENCE ID NO: 1, or a functional variant thereof, for use in treating or preventing vascular dementia or multi-infarct dementia.

The invention also relates to BSH1 enzyme of SEQUENCE ID NO: 1, or a functional variant thereof, for use in treating or preventing hypertension.

The invention also relates to BSH1 enzyme of SEQUENCE ID NO: 1, or a functional variant thereof, for use in treating or preventing a disease or condition associated with local gastrointestinal inflammatory disease such as Crohn's disease and ulcerative colitis.

The invention also relates to BSH1 enzyme of SEQUENCE ID NO: 1, or a functional variant thereof, for use in treating or preventing gastrointestinal cancer.

The invention also relates to BSH1 enzyme of SEQUENCE ID NO: 1, or a functional variant thereof, for use in treating or preventing irritable bowel syndrome (IBS).

The invention also relates to BSH1 enzyme of SEQUENCE ID NO: 1, or a functional variant thereof, for use in treating or preventing diarrhoea associated with dysregulated microbiota.

The invention also relates to an isolated bacteria selected from the group consisting of:

a strain of Lactobacillus johnsonii, comprising a 16S ribosomal RNA sequence of SEQUENCE ID NO: 8, and expressing a BSH1 enzyme having a sequence of SEQUENCE ID NO: 7;
a strain of Lactobacillus salivarius comprising a 16S ribosomal RNA sequence of SEQUENCE ID NO: 4, and expressing a BSH1 enzyme having a sequence of SEQUENCE ID NO: 3;
a strain of Lactobacillus salivarius comprising a 16S ribosomal RNA sequence of SEQUENCE ID NO: 6, and expressing a BSH1 enzyme having a sequence of SEQUENCE ID NO: 5;
a strain of Lactobacillus salivarius comprising a 16S ribosomal RNA sequence of SEQUENCE ID NO: 10, and expressing a BSH1 enzyme having a sequence of SEQUENCE ID NO: 9;
a strain of Lactobacillus salivarius comprising a 16S ribosomal RNA sequence of SEQUENCE ID NO: 12, and expressing a BSH1 enzyme having a sequence of SEQUENCE ID NO: 11;
a strain of Lactobacillus salivarius comprising a 16S ribosomal RNA sequence of SEQUENCE ID NO: 14, and expressing a BSH1 enzyme having a sequence of SEQUENCE ID NO: 13;
a strain of Lactobacillus salivarius comprising a 16S ribosomal RNA sequence of SEQUENCE ID NO: 16, and expressing a BSH1 enzyme having a sequence of SEQUENCE ID NO: 15;
a strain of Lactobacillus salivarius comprising a 16S ribosomal RNA sequence of SEQUENCE ID NO: 18, and expressing a BSH1 enzyme having a sequence of SEQUENCE ID NO: 17;
a strain of Lactobacillus salivarius comprising a 16S ribosomal RNA sequence of SEQUENCE ID NO: 20, and expressing a BSH1 enzyme having a sequence of SEQUENCE ID NO: 19;
a strain of Lactobacillus salivarius comprising a 16S ribosomal RNA sequence of SEQUENCE ID NO: 22, and expressing a BSH1 enzyme having a sequence of SEQUENCE ID NO: 21;
a strain of Lactobacillus salivarius comprising a 16S ribosomal RNA sequence of SEQUENCE ID NO: 24, and expressing a BSH1 enzyme having a sequence of SEQUENCE ID NO: 23;
a strain of Lactobacillus salivarius comprising a 16S ribosomal RNA sequence of SEQUENCE ID NO: 26, and expressing a BSH1 enzyme having a sequence of SEQUENCE ID NO: 25;
a strain of Lactobacillus salivarius comprising a 16S ribosomal RNA sequence of SEQUENCE ID NO: 28, and expressing a BSH1 enzyme having a sequence of SEQUENCE ID NO: 27;
a strain of Lactobacillus salivarius comprising a 16S ribosomal RNA sequence of SEQUENCE ID NO: 30, and expressing a BSH1 enzyme having a sequence of SEQUENCE ID NO: 29;
a strain of Lactobacillus salivarius comprising a 16S ribosomal RNA sequence of SEQUENCE ID NO: 32, and expressing a BSH1 enzyme having a sequence of SEQUENCE ID NO: 31;
a strain of Lactobacillus salivarius comprising a 16S ribosomal RNA sequence of SEQUENCE ID NO: 34, and expressing a BSH1 enzyme having a sequence of SEQUENCE ID NO: 33;
a strain of Lactobacillus salivarius comprising a 16S ribosomal RNA sequence of SEQUENCE ID NO: 36, and expressing a BSH1 enzyme having a sequence of SEQUENCE ID NO: 35;
a strain of Staphylococcus epidermidis, comprising a 16S ribosomal RNA sequence of SEQUENCE ID NO: 38, and expressing a BSH1 enzyme having a sequence of SEQUENCE ID NO: 37; and
a strain of Streptococcus salivarius, comprising a 16S ribosomal RNA sequence of SEQUENCE ID NO: 40, and expressing a BSH1 enzyme having a sequence of SEQUENCE ID NO: 39.

Typically, the Lactobacillus strains are isolated from pigs, typically pig faeces. Suitably, the Streptococcus and Staphylococcus strains are isolated from human faeces, preferably infant human faeces.

The bacteria employed in the methods of the invention are typically selected from the isolated bacteria of the invention.

### DEFINITIONS

SEQUENCE ID NO: 1 and 2 are the amino acid, and nucleic acid, sequences, respectively, of BSH1 enzyme from Lactobacillus salivarius JCM1046

SEQUENCE ID NO: 1: JCM1046 BSH1 AA Sequence (ACL98194):

| | |
|---|---|
| 1 | mctaitlngn snyfgrnldl dfsygeqvii tpaeyefkfr kekaiknhks ligvgivana |
| 61 | yplyfdaine dglgmaglnf pgnayysdal endkdnitpf efipwilrqc sdvnearnlv |
| 121 | erinlinlsf seqlplaglh wliadreksi vvevtksgvh iydnpigvlt nnpefnyqmy |
| 181 | nlnkyrnlsi stpqntfsds vdlkvdgtgf ggiglpgdvs pesrfvraaf sklnsskgtt |
| 241 | veeditqffh ilgtveqikg vnktesgkee ytvysncydl dnktlyytty enrqivavtl |
| 301 | nedkngngli aypferkqvi nkln |

SEQUENCE ID NO: 2: JCM1046 BSH1 Gene Sequence (FJ591081):

| | |
|---|---|
| 1 | atgtgtacag caattacttt aaatggtaat agtaattatt ttggaagaaa tttagatttg |
| 61 | gatttttcat atggcgagca ggtaatcatt actccggctg agtatgagtt taaatttaga |
| 121 | aaggaaaaag ctataaagaa tcataaatca ttgataggtg ttggaattgt cgctaacgct |
| 181 | tacccattgt attttgatgc tattaatgag gatggactag gaatggcagg attgaatttt |
| 241 | cctggaaatg catattatag cgatgcttta gagaatgata aagataatat tacgccgttc |
| 301 | gagtttattc catggattct gagacagtgt agcgatgtta atgaagcaag aaatttagtt |
| 361 | gaaagaataa atctcattaa tcttagtttt agcgaacaat tacctttagc agggttacat |
| 421 | tggttaattg cagatagaga aaaatccatt gtagtagaag taactaaatc tggcgtacat |
| 481 | atttatgata atccaattgg agtattgact aataatccgg aatttaatta tcagatgtac |
| 541 | aatctgaata aatatcgcaa cttatctatc agtacaccac aaaatacatt ctcagatagc |
| 601 | gtggatttaa aagtagacgg taccggtttt ggtggtattg gcttaccagg cgatgtatct |
| 661 | cccgaatctc gttttgtgag agctgctttt agcaagttaa attcaagtaa agggacgacc |
| 721 | gtagaagaag atattactca gttttttcat atactaggga cagtagaaca gataaagggc |
| 781 | gttaataaga cagaatcagg aaaagaagaa tatactgtat attcgaattg ttatgatttg |
| 841 | gacaacaaga cgttatatta tacaacctat gaaaatagac aaatagtagc tgttacttta |
| 901 | aatgaagata agaatggtaa tgggttaatt gcatatccat ttgaaagaaa acaagtaata |
| 961 | aataagttga attaa |

Lactobacillus salivarius JCM1046 was obtained from the Korean Collection of Type Cultures, KCTC 3156 (open repository).

The term “functional variant thereof” should be understood to mean a bacterial BSH enzyme having at least 60% sequence identity with SEQUENCE ID NO: 1, and which is capable of displaying an ability to significantly decongugate bile acids in vitro as determined by the chemical analysis assays described below (ninhydrin assay and UPLC-MS analysis). Non functional variants lack the ability to significantly deconjugate bile acids in these analyses. In a preferred embodiment, the functional variant is capable of altering expression of loci associated with immune function, cholesterol transport, and lipid transport and synthesis, relative to the E. coli control, when expressed in the ileum of a mouse according to the methods described below. Suitably, the functional variant is capable of altering (increasing) expression of the gene encoding the hormone adipopnectin, the gene encoding the Angiopoietin-4, and preferably both, relative to the E. coli control, when expressed in the liver of a mouse according to the methods described below. Preferably, the functional variant is capable of regulating major metabolic pathways involved in triglyceride biosynthesis, bile synthesis, and fatty acid transport and synthesis, relative to the E. coli control, when expressed in the liver of a mouse according to the methods described below.

Preferably, the functional variant of the BSH1 enzyme of SEQUENCE ID NO: 1 has at least 90%, 91%, 92%, 93%, 94%, 95%, 96%, 97%, 98% or 99% sequence identity with SEQUENCE ID NO: 1. Thus, for example, the term should be taken to include enzymes that are altered in respect of one or more amino acid residues, for example 1, 2, 3, 4, 5, 6, 7, 8, 9, or 10 amino acids compared with the BSH1 enzyme of SEQUENCE ID NO: 1. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition and substitution with natural and modified amino acids is envisaged. The variant may have conservative amino acid changes, wherein the amino acid being introduced is similar structurally, chemically, or functionally to that being substituted. Typically, the functional variant is an ortholog or paralog of BSH1 of SEQUENCE ID NO: 1. The term sequence identity comprises both sequence identity and similarity, i.e. a polypeptide sequence that shares 90% amino acid identity with SEQ ID NO: 1 is one in which any 90% of aligned residues are either identical to, or conservative substitutions of, the corresponding residues in SEQ ID NO: 1. The term “variant” is also intended to include chemical derivatives of the BSH1 enzyme of SEQUENCE ID NO: 1, i.e. where one or more residues of is chemically derivatized by reaction of a functional side group. Also included within the term variant are functional variant molecules in which naturally occurring amino acid residues are replaced with amino acid analogues. Details of amino acid analogues will be well known to those skilled in the art.

Examples of bacteria that express functional variants of BSH1 of SEQUENCE ID NO: 1 are Strains APC1484 to APC1502 described in Tables 1, 2 and 3 below. All of the strains are available within the Alimentary Pharmabiotic Centre (APC) culture collection, University College Cork, Cork, Ireland (http://www.ucc.ie/research/apc/content/)

TABLE 1

| | | | | | | Seq ID | | |
|---|---|---|---|---|---|---|---|---|
| | | | | | | No: 1 | | |
| | | | | | | BSH | | |
| Culture | | PCR | Length of | Nearest | | JCM1046 | Nearest | |
| Collection | Origin | Length | sequence | homology | % | % | homology | % |
| designation | Source | (nt) | (nt) | BSH | Homology | Identity | to rRNA | Identity |
| APC1484 | Pig | BSH1 975 | BSH1 934 | Lactobacillus | 98 | 96 | Lactobacillus | 99 |
| SEQ ID 3, 4 | | rRNA 370 | SEQ ID 3 | salivarius | | | salivarius: | |
| | | | rRNA 302 | strain | | | JCM 8665 | |
| | | | | CCUG44481 | | | | |
| APC1485 | Pig | BSH1 975 | BSH1 917 | Lactobacillus | 97 | 96 | Lactobacillus | 99 |
| SEQ ID 5, 6 | | SEQ ID 5 | rRNA 301 | salivarius | | | salivarius: | |
| | | rRNA 370 | | strain | | | JCM 8665 | |
| | | SEQ ID 6 | | CCUG44481 | | | | |
| APC1486 | Pig | BSH1 975 | BSH1 927 | Lactobacillus | 97 | 96 | Lactobacillus | 94 |
| SEQ ID 7, 8 | | rRNA 370 | rRNA 198 | salivarius | | | johnsonii | |
| | | | | strain | | | C37An8 | |
| | | | | CCUG44481 | | | | |
| APC1487 | Pig | BSH1 975 | BSH1 867 | Lactobacillus | 97 | 96 | Lactobacillus | 99 |
| SEQ ID 9, | | rRNA 370 | rRNA 319 | salivarius | | | salivarius: | |
| 10 | | | | strain | | | JCM 8665 | |
| | | | | CCUG44481 | | | | |
| APC1488 | Pig | BSH1 975 | BSH1 883 | Lactobacillus | 99 | 96 | Lactobacillus | 96 |
| SEQ ID 11, | | rRNA 370 | rRNA 283 | salivarius | | | salivarius: | |
| 12 | | | | strain | | | JCM 8665 | |
| | | | | CCUG44481 | | | | |
| APC1489 | Pig | BSH1 975 | BSH1 927 | Lactobacillus | 99 | 96 | Lactobacillus | 98 |
| SEQ ID 13, | | rRNA 370 | rRNA 294 | salivarius | | | salivarius: | |
| 14 | | | | strain | | | JCM 8665 | |
| | | | | CCUG44481 | | | | |
| APC1490 | Pig | BSH1 975 | BSH1 929 | Lactobacillus | 99 | 96 | Lactobacillus | 99 |
| SEQ ID 15, | | rRNA 370 | rRNA 310 | salivarius | | | salivarius: | |
| 16 | | | | strain | | | JCM 8665 | |
| | | | | CCUG44481 | | | | |
| APC1491 | Pig | BSH1 975 | BSH1 915 | Lactobacillus | 99 | 96 | Lactobacillus | 99 |
| SEQ ID 17, | | rRNA 370 | rRNA 308 | salivarius | | | salivarius: | |
| 18 | | | | strain | | | JCM 8665 | |
| | | | | CCUG44481 | | | | |
| APC1492 | Pig | BSH1 975 | BSH1 907 | Lactobacillus | 99 | 97 | Lactobacillus | 99 |
| SEQ ID 19, | | rRNA 370 | rRNA 298 | salivarius | | | salivarius: | |
| 20 | | | | strain | | | JCM 8665 | |
| | | | | CCUG44481 | | | | |
| APC1493 | Pig | BSH1 975 | BSH1 926 | Lactobacillus | 98 | 95 | Lactobacillus | 99 |
| SEQ ID 21, | | rRNA 370 | rRNA 306 | salivarius | | | salivarius: | |
| 22 | | | | strain | | | JCM 8665 | |
| | | | | CCUG44481 | | | | |
| | | | | | | Seq ID | | |
| | | | | | | No: 1 | | |
| | | Predicted | | | | BSH | | |
| | | PCR | Actual | Nearest | | JCM1046 | Nearest | |
| Culture | Origin | length | Length | homology | % | % | homology to | % |
| Designation | Source | (nt) | (nt) | BSH | Homology | Identity | rRNA | Identity |
| APC1494 | Pig | BSH1 975 | BSH1 927 | Lactobacillus | 97 | 95 | Lactobacillus | 99 |
| SEQ ID 23, | | rRNA 370 | rRNA 320 | salivarius | | | salivarius: | |
| 24 | | | | strain | | | JCM 8665 | |
| | | | | CCUG44481 | | | | |
| APC1495 | Pig | BSH1 975 | BSH1 937 | Lactobacillus | 99 | 96 | Lactobacillus | 98 |
| SEQ ID 25, | | rRNA 370 | rRNA 62 | salivarius | | | salivarius | |
| 26 | | | | strain | | | strain CI2 | |
| | | | | CCUG44481 | | | | |
| APC1496 | Pig | BSH1 975 | BSH1 888 | Lactobacillus | 97 | 96 | Lactobacillus | 99 |
| SEQ ID 27, | | rRNA 370 | rRNA 317 | salivarius | | | salivarius: | |
| 28 | | | | strain | | | JCM 8665 | |
| | | | | CCUG44481 | | | | |
| APC1497 | Pig | BSH1 975 | BSH1 869 | Lactobacillus | 99 | 97 | Lactobacillus | 99 |
| SEQ ID 29, | | rRNA 370 | rRNA 311 | salivarius | | | salivarius: | |
| 30 | | | | strain | | | JCM 8665 | |
| | | | | CCUG44481 | | | | |
| APC1498 | Pig | BSH1 975 | BSH1 913 | Lactobacillus | 98 | 96 | Lactobacillus | 99 |
| SEQ ID 31, | | rRNA 370 | rRNA 321 | salivarius | | | salivarius: | |
| 32 | | | | strain | | | JCM 8665 | |
| | | | | CCUG44481 | | | | |
| APC1499 | Pig | BSH1 975 | BSH1 838 | Lactobacillus | 97 | 97 | Lactobacillus | 99 |
| SEQ ID 33, | | rRNA 370 | rRNA 328 | salivarius | | | salivarius: | |
| 34 | | | | strain | | | JCM 8665 | |
| | | | | CCUG44481 | | | | |
| APC1500 | Pig | BSH1 975 | BSH1 923 | Lactobacillus | 98 | 96 | Lactobacillus | 89 |
| SEQ ID 35, | | rRNA 370 | rRNA 38 | salivarius | | | salivarius: | |
| 36 | | | | strain | | | JCM 8665 | |
| | | | | CCUG44481 | | | | |

TABLE 2

| | | PCR | Sequence | | | JCM1046 |
|---|---|---|---|---|---|---|
| Strain | Origin | Length | Length | Nearest homology | % | % |
| Designation | Source | (nt) | (nt) | bsh | Homology | Identity |
| APC1501 | Human | BSH1 975 | BSH1 723 | Lactobacillus salivarius | 97 | 97 |
| SEQ ID 37 | | | | JCM1046 | | |
| APC1502 | Human | BSH1 975 | BSH1 760 | Lactobacillus salivarius | 98 | 96 |
| SEQ ID 39 | | | | strain CCUG44481 | | |

TABLE 3

| | | Length it | Length of | | % |
|---|---|---|---|---|---|
| Origin | Origin | should | sequence | Nearest homology | Iden- |
| Plate | Source | be (bp) | (bp) | to rRNA | tity |
| APC1501 | Human | rRNA | rRNA | Staphylococcus | 99 |
| SEQ ID | | 370 | 313 | epidermidis | |
| 38 | | | | strain IHB B 12019 | |
| APC1502 | Human | rRNA | rRNA | Streptococcus | 99 |
| SEQ ID | | 370 | 307 | salivarius partial | |
| 40 | | | | 16S rRNA gene, | |
| | | | | isolate OCAT30 | |

The BSH1 gene sequences and 16s rRNA sequences for the strains referenced in Tables 1-3 are provided in the Appendix below.

Proteins (including variants thereof) of and for use in the invention may be generated wholly or partly by chemical synthesis or by expression from nucleic acid. The proteins and peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984)).

In this specification, the term “elevated expression” as applied to the level of expression of the active of the invention in a bacterial host should be understood to mean an expression level that is greater than the expression level of BSH1 in the genetically modified Escherichia coli strain MG1655 (ECBSH1).

In this specification, the term “probiotic” as applied to a bacteria should be understood to mean a live microorganism that confers a health benefit on the host.

In this specification, the term “obesity” should be understood to mean a body mass index of greater than 30 kg/m2.

In this specification, the term “hypercholesteremia” should be understood to mean total cholesterol of greater than 5 mmol/L, and low-density lipoprotein cholesterol (LDL) of greater than 3 mmol/L. For people at high risk of cardiovascular disease, the recommendation for total cholesterol is 4 mmol/L or less, and 2 mmol/L or less for LDL.

In this specification, the term “metabolic disorder” should be understood to mean a disease or condition that disrupts normal metabolism in a mammal. Examples include: pre-diabetes, diabetes; Type-1 diabetes; Type-2 diabetes; metabolic syndrome; obesity; diabetic dyslipidemia; hyperlipidemia; hypertension; hypertriglyceridemia; hyperfattyacidemia; hypercholerterolemia; MODY; HNF1A-MODY; and hyperinsulinemia. Preferably, the metabolic disorder is selected from MODY; HNF1A-MODY; pre-diabetes, and diabetes (including Type-1 diabetes or Type-2 diabetes).

The invention also relates to a recombinant vector comprising a nucleic acid encoding a BSH1 enzyme of SEQUENCE ID NO: 1, or a functional variant thereof, optionally under the control of a constitutive promotor. Typically, the nucleic acid is cloned into a recombinant vector (for example a plasmid) which is capable of replicating in the host bacteria. Typical plasmids contain, in addition to the cloned insert, a selection gene (i.e. antibiotic resistance, a dye etc) and an origin of replication effective in the host bacterium. The plasmid may also comprise regulatory sequences, for example promoters, terminators and/or enhancers. Examples of such vectors include pBKminiTn7GM2 (Koch, B., Jensen, L. E., and Nybroe, O. (2001). A panel of Tn7-based vectors for insertion of the gfp marker gene or for delivery of cloned DNA into Gram-negative bacteria at a neutral chromosomal site. J Microbiol Methods 45, 187-195) or pNZ44 (McGrath, S., Fitzgerald, G. F., and van Sinderen, D. (2001). Improvement and optimization of two engineered phage resistance mechanisms in Lactococcus lactis. Appl Environ Microbiol 67, 608-616.)

The nucleic acid may also be cloned into an integrative cassette suitable for integration into the genome of suitable host bacteria. Such an integrative cassette typically comprises a nucleic acid encoding the BSH1 enzyme of SEQUENCE ID NO: 1, or a functional variant thereof, linked to (or flanked by) one or several sequences allowing integration, preferably site-specific integration. Such sequences may be for instance nucleic acid sequences homologous to a targeted region of the genome, allowing integration through crossing over. Various techniques can be used to insert a nucleic acid into a host bacteria, for example through natural transformation or electroporation.

The host bacteria suitable for cloning the active of the invention may be selected from any host bacteria known to a person skilled in the art such as, for example, Bifidobactrium (B. adolescentis, B. animalis, B. breve, B. infantis, B. longum, B. sp), Lactobacillus (L, acidophilus, L. casei, L. feermentus, L. gasseri). Preferably, the host bacteria is a probiotic bacteria.

In the specification, the term “mammal” or “individual” as employed herein should be taken to mean a human; however it should also include higher mammals for which the method, prophylaxis, therapy or use of the invention is practicable, for example, pigs. The term “animal” should be understood to include any animal including humans.

In this specification, the term “administering” should be taken to include any form of delivery that is capable of delivering the enzyme or bacteria, including local delivery, intravenous delivery, oral delivery, intranasal delivery, intramuscular delivery, intrathecal delivery, transdermal delivery, inhaled delivery and topical delivery. Methods for achieving these means of delivery will be well known to those skilled in the art of drug delivery.

In this specification, the term “pharmaceutical composition” should be taken to mean compositions comprising a therapeutically effective amount of the active of the invention, that in one embodiment are produced in-situ in the composition by a bacterial strain, and a pharmaceutically acceptable carrier or diluent. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the bacterial strain and/or active of the invention is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

“Effective amount” refers to the amount or dose of the active of the invention upon single or multiple dose administration to the patient, which provides the desired effect in the patient under treatment. An effective amount can be readily determined by the attending diagnostician, as one skilled in the art, by the use of known techniques and by observing results obtained under analogous circumstances. In determining the effective amount or dose of enzyme or bacterial strain expressing the enzyme administered, a number of factors are considered by the attending diagnostician, including, but not limited to: the species of mammal; its size, age, and general health; the specific disease involved; the degree of or involvement or the severity of the disease; the response of the individual patient; the mode of administration; the bioavailability characteristics of the preparation administered; the dose regimen selected; the use of concomitant medication; and other relevant circumstances.

The term “comestible product” should be understood to include products that are intended to be consumed by ingestion by humans or animals, such as foods and drinks. In particular, the comestible product is a food or drink product intended for consumption by humans, for example a fermented product or a diary product, especially a fermented dairy product such as a yoghurt.

### BRIEF DESCRIPTION OF THE FIGURES

FIG. 1. Expression of cloned BSH in E. coli MG1655 and activity in murine gallbladder bile in vitro. (A) Cloning strategy for expression of BSH enzymes in E. coli MG1655. (B) Ninhydrin assay showing the release of taurine from conjugated bile acids as an index of BSH activity. *** P<0.001 (n=5 per group). (C) Heat maps summarizing UPLC-MS analysis of individual bile acids in murine bile in vitro following 90 minute exposure to E. coli MG1655 (EC) or clones expressing BSH activities ECBSH1 and ECBSH2 or empty vector control ECpNZ44. Results represent analysis of 3 biological replicates.

FIG. 2. Alterations of host bile acid signatures through gastrointestinal expression of cloned BSH in gnotobiotic mice. (A) Total plasma bile acids (assessed by UPLC-MS) in germ free (GF) mice, mice mono-colonised with E. coli (EC) or E. coli chromosomally expressing BSH (ECBSH1 and ECBSH2) and conventionalised mice (CONV-D). *** P<0.001, ** P<0.05 relative to E. coli controls (n=5 per group). (B) Total tauroconjugated bile acids (assessed by UPLC-MS) in plasma of germ free (GF) mice, mice mono-colonised with EC or ECBSH1 or ECBSH2 and CONV-D mice. *** P<0.001, ** P<0.05 relative to E. coli controls (n=5 per group). (C) Total tauroconjugated murocholic acid moieties in plasma of germ free (GF) mice, mice mono-colonised with EC or ECBSH1 or ECBSH2 and CONV-D mice. ** P<0.0001, * P<0.005 relative to E. coli (EC) controls (n=5 per group). (D) Total unconjugated murocholic acid moieties in plasma of germ free (GF) mice, mice mono-colonised with EC or ECBSH1 or ECBSH2 and CONV-D mice. ** P<0.0001, * P<0.005 relative to E. coli (EC) controls (n=5 per group). (E) Influence of gastrointestinal BSH expression upon cyp7a expression in the livers of monocolonised mice. cyp7a transcript was measured by quantitative RT-PCR (n=5 per group). Data are presented as means±SEM, *=vs. GF, #=vs. ECBSH1, compared to respective control, ** P<0.01, * P<0.05. (F) Total levels of secondary and tertiary bile acids in plasma of germ free (GF) mice, mice mono-colonised with EC or ECBSH1 or ECBSH2 and CONV-D mice. *** P<0.001 relative to E. coli controls (n=5 per group).

FIG. 3. BSH expression in the GI tract of gnotobiotic mice significantly alters gene expression patterns in ileal and hepatic tissue. Microarray analysis of ileal and liver tissue from germ free (GF) mice, conventionalised (CONV-D) mice or animals monocolonised with EC, ECBSH1 or ECBSH2. Shown are heat maps representing gene expression profiles of selected genes that were significantly (P<0.05) altered through BSH1 expression in our system. Pathways related to lipid digestion and absorption, circadian rhythm, adiposignalling and immune homeostasis were most significantly affected as determined by pathway analysis and are shown here. (n=5 mice per group). Schematic indicates key transcriptional changes affected by BSH1 expression. Genes increased in ECBSH1 colonised mice relative to EC colonised mice are indicated in red, genes decreased in ECBSH1 colonised mice relative to EC colonised mice are indicated in blue.

FIG. 4. Gastrointestinal expression of cloned BSH in conventional mice alters plasma bile acid profiles. Mice were provided with streptomycin (5 mg ml⁻¹) ad libitum in drinking water in order to promote stable high-level colonisation of the host E. coli MG1655 Strep^{R} strain as described previously (Chang et al., 2004). (A) Total plasma bile acids (assessed by UPLC-MS) in conventional mice (not-treated, NT), conventional mice with antibiotic only (Ab), mice colonised with E. coli (EC) or E. coli expressing BSH (ECBSH1 or ECBSH2). *** P<0.0002 relative to E. coli controls (n=5 per group). (B) Total tauroconjugated plasma bile acids (assessed by UPLC-MS) in NT mice, Ab-treated mice, mice colonised by EC, ECBSH1 or ECBSH2. *** P<0.0002 relative to E. coli controls (n=5 per group). (C) Relative proportions of primary bile acids (pBAs), secondary and tertiary bile acids (stBAs) and tauroconjugated bile acids (T-CBAs) in the plasma of uncolonised NT and Ab mice and mice colonised by EC, ECBSH1 or ECBSH2. (D) Total tauroconjugated murocholic acid moieties in plasma of conventionally raised NT mice, Ab-treated mice, mice colonised by EC, ECBSH1 or ECBSH2. ** P<0.0001, * P<0.005 relative to E. coli (EC) controls (n=5 per group). (E) Total unconjugated murocholic acid moieties in plasma of NT mice, Ab-treated mice, mice colonised by EC, ECBSH1 or ECBSH2. ** P<0.0001, * P<0.005 relative to E. coli (EC) controls (n=5 per group).

FIG. 5. Gastrointestinal expression of cloned BSH in conventionally raised mice reduces weight gain, serum cholesterol and liver triglycerides. (A) Average weight gain over time measured in grams following colonisation of mice with EC or ECBSH1. Data represent antibiotic-treated mice (solid circles), EC colonised mice (solid squares) or ECBSH1 colonised mice (solid triangles) with weight gain monitored over 10 weeks. (B) Weight of total excised fat from mice undergoing Ab treatment alone or mice colonised by EC, ECBSH1 or ECBSH2. ** P<0.0085 relative to the EC colonised control (n=5 per group). (C) Levels of LDL cholesterol in plasma in mice colonised by EC, ECBSH1, ECBSH2 or control uncolonised mice. LDL cholesterol levels were measured using Cholesterol quantification kit BioVision, CA, USA. * P<0.05 relative to controls as indicated. (n=5 per group). (D) Levels of liver triglycerides in mice colonised by EC, ECBSH1, ECBSH2 or antibiotic treated controls (Ab). Liver triglycerides were measured using triglyceride liquid stable reagent (Thermoscientific). * P<0.05. (n=5 per group).

FIG. 6. Gastrointestinal expression of BSH influences gene expression patterns in ileal tissue in conventional mice. Mice given streptomycin were colonised by E. coli MG1655 Strep^{R} as outlined in our model system. Gene expression patterns of selected genes were examined using qRT-PCR in ileal tissue in mice colonised by EC, ECBSH1, ECBSH2 and in uncolonised animals. (n=5 per group). Statistical significance was determined using ANOVA. Data are presented as means±SEM, *=vs. GF, #=vs. ECBSH1, $=vs. EC compared to respective control, *** P<0.001, ** P<0.01, *P<0.05

FIG. 7. Markerlynx Principal Component Analysis (PCA) using a bile acid database and pareto template for orthogonal partial least square discriminant analysis (OPLS DA analysis) and extended statistics applied to Swiss Webster plasma Bile acids (BAs). The noise levels were 15%, 24% and 34% for comparative analysis of A. GF (Germ Free) treated and EC (E. coli MG1655 StrepR) treated; B. EC treated and ECBSH1 (E. coli MG1655 StrepR carrying BSH1) treated and C. EC treated and ECBSH2 (E. coli MG1655 StrepR carrying BSH2) treated respectively (n=5). Three technical replicates were read for each sample and the markers were normalized relative to the level of deuterated Internal Standards with which samples were spiked pre extraction.

FIG. 8. qPCR confirmation of selected microarray mRNA expression targets. Selected Genes of Interest (GOIs) identified from the microarray analysis were subjected to qRT-PCR for independent confirmation. qPCR data is expressed as relative expression compared to beta-actin housekeeper control in ileum or liver tissue samples mice in the respective treatment group. Treatment groups were germ free (GF) mice, conventionalised (CONV-D) mice or animals monocolonised with EC, ECBSH1 or ECBSH2. Statistical significance was determined using ANOVA. Data are presented as means±SEM, n=5/group. *=vs GF, #=vs BSH1, $=vs EC, *** P<0.001, ** P<0.01, *P<0.05, compared to respective control.

FIG. 9 E. coli colonization in the gastrointestinal tract of conventional C57Bl/6J mice administered streptomycin ad libitum. E. coli was enumerated by standard plate counts from faeces on the days indicated.

FIG. 10 (Supplementary FIG. S4) BSH1 activity lowers weight gain in mice fed a high fat chow (45% calories from fat Research Diets (HFD)) or normal chow (10% calories from fat Research Diets (LFD)). Experimental design as per FIG. 5A. B. Weight of total excised fat from mice undergoing Ab treatment alone or mice colonised with EC, ECBSH1 or ECBSH2. Data from mice fed normal chow (LFD) or high fat diet (HFD). * P<0.05 relative to the EC dataset in each case. ECBSH1 colonisation lowers C plasma cholesterol and D liver triglycerides in mice fed either a LFD or a HFD. * indicates P<0.05 relative to EC colonised mice.

FIG. 11 Absolute levels of A plasma and B liver cytokines in conventional C57Bl/6J mice colonised by EC in our model system as measured by Mesoscale Discovery assay.

FIG. 12 Figure outlining relative bile acid modifications by strains P003, P005 and JCM1046 compared to untreated human bile as determined by UPLC-MS:

### DETAILED DESCRIPTION OF THE INVENTION

### Experimental Procedures

### Bile Salt Hydrolase Cloning.

Bile salt hydrolases from Lactobacillus salivarius strains (Fang et al., 2009) were cloned independently into pBKminiTn7GM2 (Koch et al., 2001) under the control of the P44 promoter (McGrath et al., 2001) using splicing by overlap extension (SOE) PCR. Transposon integration was carried out as described previously (Koch et al., 2001). PCR downstream from the glmS region confirmed constructions as did sequence analysis (GATC Biotech).

### Bile Salt Activity Assay.

EC, ECBSH1 and ECBSH2 were examined for their ability to deconjugate bile in vitro using the ninhydrin assay for free taurine (Lipscomb et al., 2006) and by co-incubation for 90 minutes in murine gall bladder BA followed by UPLC MS analysis. Protein concentrations were measured with the Biorad Protein Assay (Biorad, Hercules, Calif.), and bovine serum albumen (BSA) (Sigma) was used as standard.

### Mice.

Germ free Swiss Webster mice were maintained in the germ-free unit in the Alimentary Pharmabiotic Centre. Monocolonisation experiments were initiated by oral dosing of appropriate strains at 1×10⁹ CFU per mouse. Monocolonised mice were housed in relevant groups in individual germ free isolators for the duration of the experiment. For analysis of conventional mice C57Bl/6J male mice were purchased from Harlan (Oxon, UK) and housed under barrier maintained conditions at University College Cork. 6 week old male C57Bl/6J mice were fasted for 24 hours and immediately supplied with Streptomycin treated drinking water (5 mg ml⁻¹ final concentration) for the duration of the experiment. After 24 hours mice were fed either a low fat diet ((n=20) 10% calories from fat Research Diets International, New Jersey, USA D12450B) or a high fat diet ((n=20) 45% calories from fat Research Diets International, New Jersey, USA D12451) for 10 weeks. These two groups were further divided into parallel groups (n=5 for each group) and were inoculated with relevant strains in PBS at 1×10⁶ CFU per mouse by oral gavage (inoculations on two consecutive days). Body weight and food intake was assessed weekly. Faecal samples were taken from each individual on a weekly basis and used for bacterial enumeration. At the end of the study mice were sacrificed and internal organs (liver, spleen, intestine) and fat pads (reproductive, renal, mesenteric and inguinal) were removed, weighed and stored at −80° C. The experiments outlined were approved by the University Animal Experimentation Ethics Committee.

### Metabolic Markers.

Mice were fasted for 5-6 hours and blood glucose was measured using a Contour glucose meter (Bayer, UK) using blood collected from the tip of the tail vein. Blood was collected by cardiac puncture and plasma was extracted. Plasma insulin concentrations were determined using an ELISA kit (Mercodia, Uppsala, Sweden), plasma and liver triglyceride levels were determined using infinity triglyceride liquid stable reagent (Thermoscientific) and cholesterol levels were determined from plasma Cholesterol quantification kit (BioVision, CA, USA). Inflammasome activation was assessed using 7-plex MesoScale Discovery Kit (Gaithersburg, Md., USA) directly from plasma and from liver extracts.

### Chemicals.

Standard C-BAs and BAs were purchased from Sigma Aldrich or Steraloids and are listed in supplementary information (Table S1). Deuterated cholic acid (D-2452) and deuterated chenodeoxycholic acid (D-2772) were purchased from CDN Isotopes Inc. HPLC-grade methanol, acetonitrile, water, ammonium acetate, ammonium formate, ammonium hydroxide, formic acid, and acetic acid and water were obtained from Fisher Scientific (Fair Lawn, N.J.). Standards were constituted as 1 mg/ml stock solutions of individual sulfated BAs were prepared in water:MeOH (1:1) and combined to a final volume of 1.0 ml in water to give a concentration of 40 mg/ml for each. Subsequent dilutions were made as necessary to create a standard curve for each bile acid.

### Bile Acid Extractions.

Bile acids were extracted from 100 μl of plasma spiked with internal standards added to 50% ice-cold methanol. The extract was mixed then centrifuged at 16,000×g for 10 minutes at 4° C. The supernatant was retained and further extracted by addition of ACN (5% NH₄OH). The resultant supernatant was dried under vacuum and reconstituted in 50% MeOH. The extracted bile acids were resuspended in 150 ml of ice cold 50% MeOH.

### Ultra Performance Liquid Chromatography Tandem Mass Spectrometry.

UPLC-MS was performed using a modified method of Swann et al. (Swann et al., 2011). 5 μL were injected onto a 50 mm T3 Acquity column (Waters Corp.) and were eluted using a 20-min gradient of 100% A to 100% B (A, water, 0.1% formic acid; B, methanol, 0.1% formic acid) at a flow rate of 400 μL/min and column temperature of 50° C. Samples were analyzed using an Acquity UPLC system (Waters Ltd.) coupled online to an LCT Premier mass spectrometer (Waters MS Technologies, Ltd.) in negative electrospray mode with a scan range of 50-1,000 m/z. Bile acids ionize strongly in negative mode, producing a prominent [M-H]− ion. Capillary voltage was 2.4 Kv, sample cone was 35 V, desolvation temperature was 350° C., source temperature was 120° C., and desolvation gas flow was 900 L/h.

PCA analysis was performed in Markerlynx (Waters) by limiting the number of elements (N, H, S, C) to be detected in individual analytes. Furthermore a template of defined known masses to allow bile acid detection only was applied to generate a table of markers and their retention time. Group Differences were detected using the pareto scaling in OPLS-DA. Here weighted averages provide a summary of the X variables. In addition, these scores of PLS-DA display the separation of the groups. The scores t[1] and t[2] summarize separating the data. The plot of t[1] vs. t[2] shows a picture of the data. The groups (types) are shown in different colours, and the separation of the groups is easily visible. Each analyte was identified according to its mass and retention time. Standard curves were then performed using known bile acids and each analyte was quantified according to the standard curve and normalized according to the deuterated internal standards.

### Microarrays.

Tissues were stored in RNA-later (Qiagen) prior to RNA extraction using the RNAeasy plus universal kit (Qiagen). Microarrays were carried out using mouse Exon ST1.0 arrays (Affymetrix) by Almac Group, Craigavon, Northern Ireland. Analysis and pathway mapping was carried out using Subio Platform software (Subio Inc) and Genesis Software. Microarray data will be deposited on the Gene Expression Omnibus website.

### Quantitative Reverse Transcriptase PCR.

qRT-PCR utilised RNA to generate cDNA. Universal ProbeLibrary (Roche) designed primers and pairs were used for qPCR with the LightCycler 480 System (Roche). The 2^{-ΔΔC} method (Livak and Schmittgen, 2001) was used to calculate relative changes in gene expression.

### Statistical Analysis.

Data for all variables were normally distributed and therefore allowed for parametric test of significance. Data is presented as mean values and their standard deviation is indicated. Statistical analysis was performed by analysis of variance and students t test.

### Isolation of Strains Expressing Seq ID No: 1 BSH Activity:

Pig samples were taken from the porcine facility in the biological services unit in UCC and human faeces was from a 2 year old female infant donor. Samples of porcine or human faeces were sieved, serially diluted (in phosphate buffered saline, PBS) and plated onto MRS plates under anaerobic conditions. Single colonies were grown anaerobically in MRS broth in 96-well plates for further characterisation. 960 putative Lactobacillus species isolates were isolated for further characterisation. Isolates were screened using PCR for the presence of BSH1 (Seq ID No: 1) based upon the presence of known regions using the following primer pairs:

(SEQ ID 41)

F1-ATGTGTACAGCAATTACTTTAAATGGTAATAGTAATTATT
and

(SEQ ID 42)

R-TTAATTCAACTTATTTATTACTTGTTTTCTTTCAAATGGA
Or

(SEQ ID 43)

F2-ATCCAATTGGAGTATTGACTAATAATCCGGAATTTAATT
and

(SEQ ID 44)

R-TTAATTCAACTTATTTATTACTTGTTTTCTTTCAAATGGA

The F1/R detects the full length BSH1 sequence whereas the F2/R primer set detects the presence of a unique 24 nt region. We sequenced BSH genes from 17 isolates from pigs (labelled as APC1484 to APC1500) and 2 isolates from human faeces (labelled APC1501 and APC1502) (see Table). We generated PCR products using 16s primers (F-DG74-AGGAGGTGATCCAACCGCA (SEQ ID 45) and R-RW01-AACTGGAGGAAGGTGGGGAT (SEQ ID 46)) which were sequenced in each case to determine the closest homologues in the NCBI database. This allowed identification of strains to species level (see Table).

### Comparison of Bile Hydrolase Activity of Isolates Using UPLC-MS:

Two porcine strains APC1486 (Lactobacillus. salivarius APC1486) and APC1488 (Lactobacillus johnsonii APC1488) and a type strain expressing Seq ID No: 1 activity (Lb. salivarius JCM1046) were incubated separately with a human bile extract (0.5% w/v in MRS broth) (obtained from clinical cholesystectomy from Cork University Hospital) for 90 mins anaerobically at 37 degrees C. Subsequently either untreated or bacterially treated human bile was subjected to chemical analysis using UPLC-MS (see below).

Standard C-BAs and BAs were purchased from Sigma Aldrich or Steraloids. Deuterated cholic acid (D-2452) and deuterated chenodeoxycholic acid (D-2772) were purchased from CDN Isotopes Inc. HPLC-grade methanol, acetonitrile, water, ammonium acetate, ammonium formate, ammonium hydroxide, formic acid, and acetic acid and water were obtained from Fisher Scientific (Fair Lawn, N.J.). Standards were constituted as 1 mg/ml stock solutions of individual sulfated BAs were prepared in water:MeOH (1:1) and combined to a final volume of 1.0 ml in water to give a concentration of 40 mg/ml for each. Subsequent dilutions were made as necessary.

### Bile Acid Extractions.

Bile acids were extracted from 100 μl of plasma added to 50% ice-cold methanol. The extract was mixed then centrifuged at 16,000×g for 10 minutes at 4° C. The supernatant was retained and further extracted by addition of ACN (5% NH4OH). The resultant supernatant was dried under vacuum and reconstituted in 50% MeOH. The extracted bile acids were resuspended in 150 ml of ice cold 50% MeOH.

### Ultra Performance Liquid Chromatography Tandem Mass Spectrometry.

UPLC-MS was performed using a modified method of Swann et al. (5). 5 μL were injected onto a 50 mm T3 Acquity column (Waters Corp.) and were eluted using a 20-min gradient of 100% A to 100% B (A, water, 0.1% formic acid; B, methanol, 0.1% formic acid) at a flow rate of 400 μL/min and column temperature of 50° C. Samples were analyzed using an Acquity UPLC system (Waters Ltd.) coupled online to an LCT Premier mass spectrometer (Waters MS Technologies, Ltd.) in negative electrospray mode with a scan range of 50-1,000 m/z. Bile acids ionize strongly in negative mode, producing a prominent [M-H]− ion. Capillary voltage was 2.4 Kv, sample cone was 35 V, desolvation temperature was 350° C., source temperature was 120° C., and desolvation gas flow was 900 L/h.

Bile acid deconjugation profiles were highly similar to those of a type strain expressing Seq ID No: 1 activity BSH activity (Lb. salivarius JCM1046) (see Figure outlining in vitro bile acid profiles) and exhibited ability to deconjugate conjugated bile acids and to generate cholic acid (CA) and chenodeoxycholic acid (CDCA) in the sample mixture.

### Deposition of Strains APC1484 to APC1502:

Strains are available upon request from the Alimentary Pharmabiotic Centre, University College Cork, Cork, Ireland (http://www.ucc.ie/research/apc/content/)

### Results

### Significant Alteration of Bile Acid Profiles in Gnotobiotic Mice Through Gastrointestinal BSH Activity

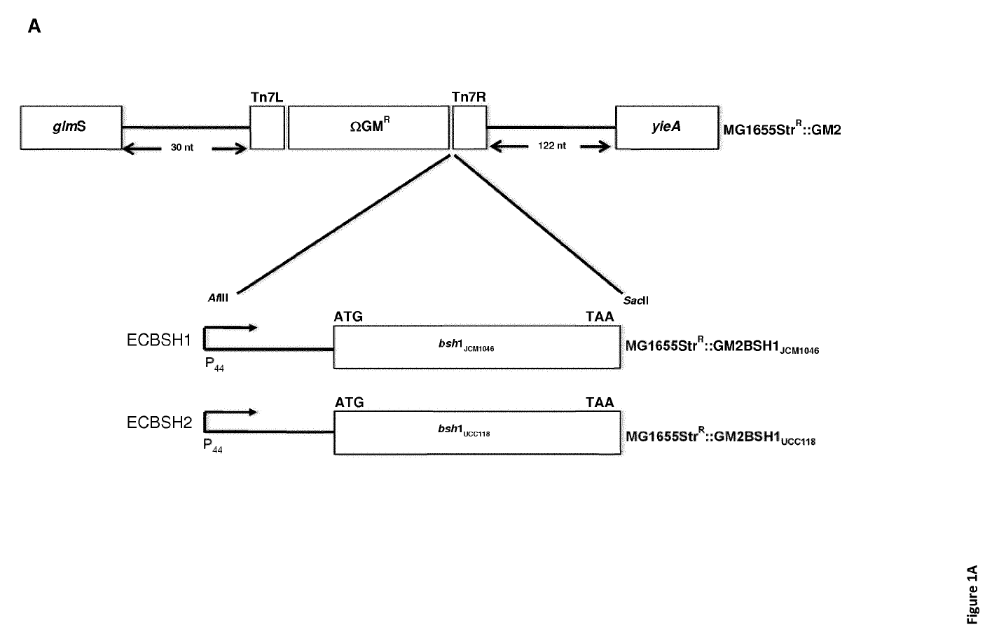

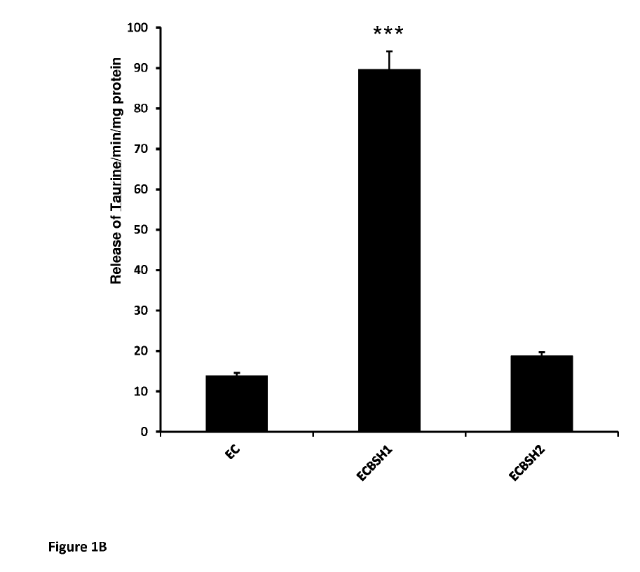

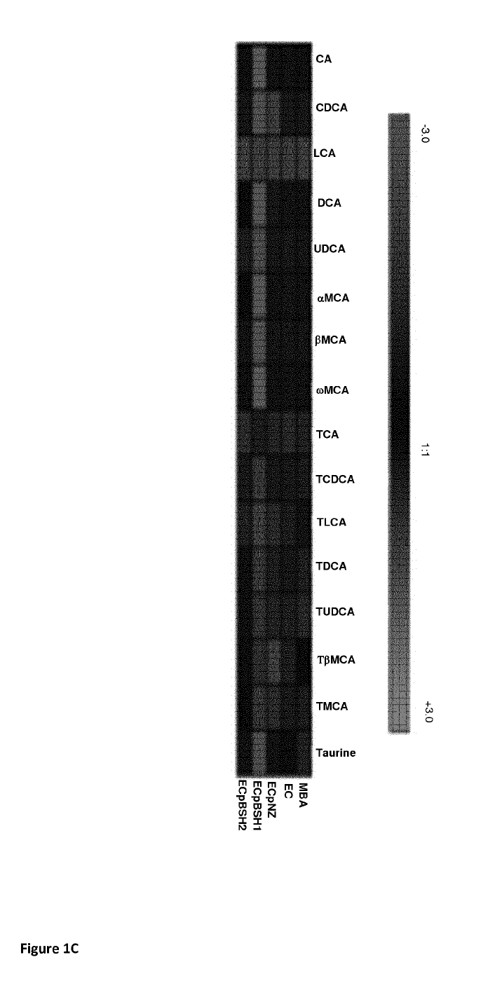

The wide variation in BSH enzymes within the gut microbiota suggests that different BSH alleles may have differing impacts upon in vivo bile metabolism and downstream responses. To compare different BSH enzymes using an isogenic delivery system, bsh genes were expressed in Escherichia coli MG1655, a K-12 strain which lacks BSH activity and colonises both conventional and germ-free (this study) mice at high levels. To achieve stable expression in long-term colonisation experiments we utilised the mini-Tn7 transposon system for the cloning of bsh genes in single copy into the region downstream of glmS in the E. coli host (FIG. 1A). bsh genes from Lactobacillus salivarius JCM1046 (herein designated as BSH1) and Lb. salivarius UCC118 (designated as BSH2) were cloned and expressed, which display defined structural differences. Both BSHs can deconjugate tauroconjugated bile acids in vitro as determined by the ninhydrin release assay (FIG. 1B) with BSH1 demonstrating the greatest efficiency in catalysing the release of taurine. E. coli alone (EC) or E. coli clones expressing BSH1 (ECBSH1) or BSH2 (ECBSH2) were exposed to ex vivo murine gallbladder bile for 90 minutes and then examined individual bile acid profiles using a sensitive ultra-performance liquid chromatography mass spec (UPLC-MS) protocol. BSH1 exhibited the greatest efficacy in generating deconjugated bile acids when measured in this in vitro system; however BSH2 also exhibited demonstrable deconjugation activity (FIG. 1C).

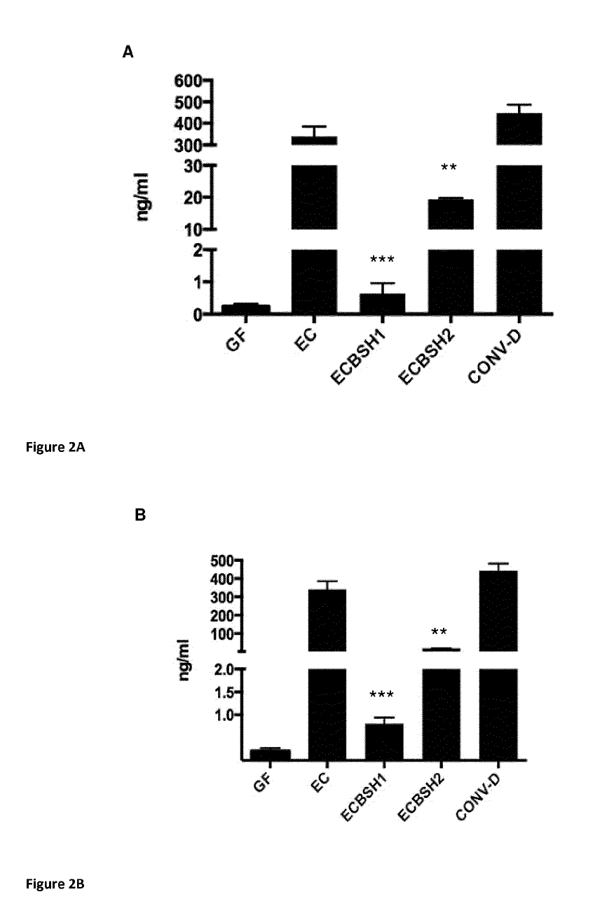

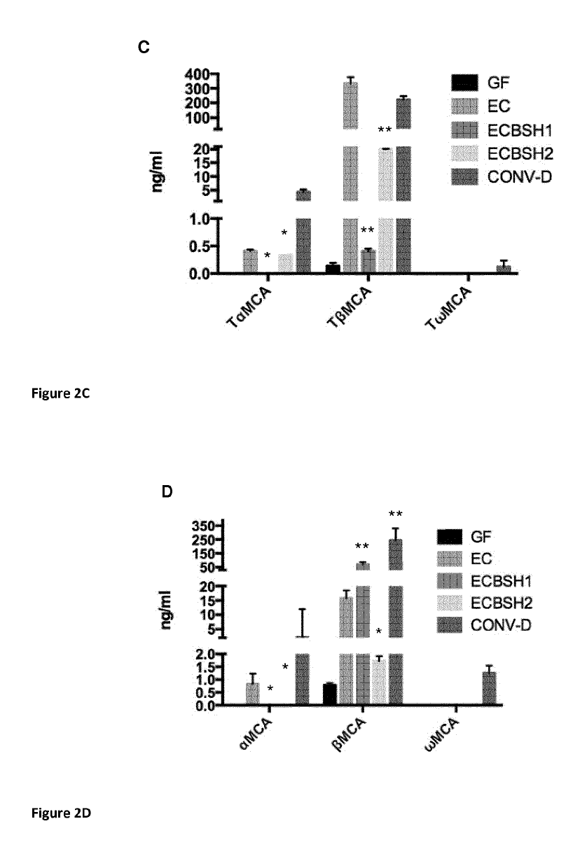

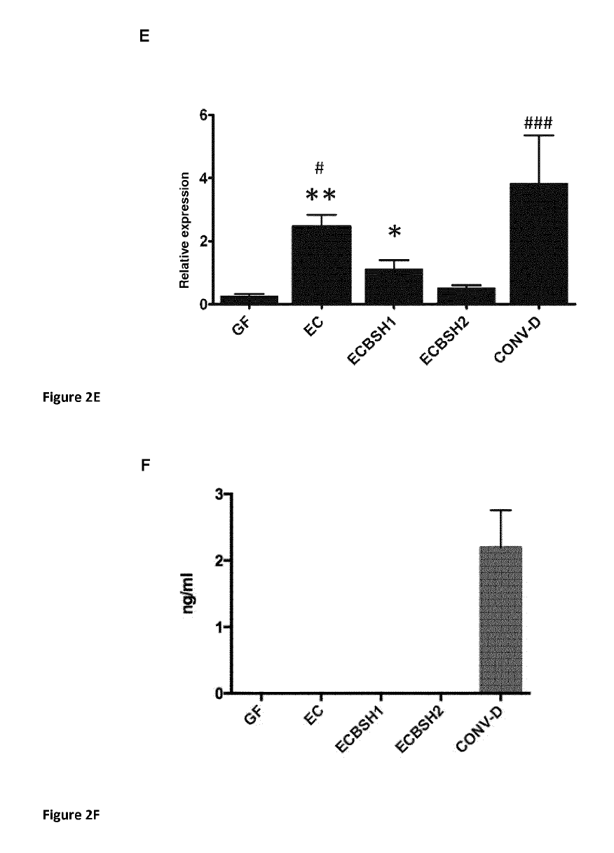

In order to analyse the physiological effects of bile hydrolysis in a controlled system which lacks extant bile modification systems, gnotobiotic mice were monocolonised with our E. coli strains expressing BSH activity (ECBSH1 or ECBSH2). Colonisation of germ-free mice with BSH⁻ E. coli MG1655 (EC) resulted in a significant elevation of total plasma bile acids to levels similar to those of conventionalised mice (CONV-D) (FIG. 2A) indicating that bacterial colonisation influences bile metabolism, regardless of BSH status. In this system BSH activity in situ resulted in a significant reduction of total plasma bile acids and a specific reduction in tauroconjugated bile acids relative to the E. coli (EC) colonised mice, demonstrating the effects of in vivo deconjugation of bile acids (FIGS. 2A and 2B). In particular, a reduction in the levels of the potent FXR-antagonist tauro-beta-murocholic acid (TbMCA) relative to EC colonised gnotobiotic mice was seen as a result of in situ BSH expression (FIGS. 2C and 2D). The findings may reflect poor enterohepatic uptake of deconjugated bile acids relative to conjugated bile acids in the ileum. However, gastrointestinal BSH activity significantly reduced expression of the hepatic gene encoding a rate limiting enzyme in the synthesis of bile acids, Cholesterol 7 alpha-hydroxylase (Cyp7a1), consistent with reduced de novo synthesis of bile acids (FIG. 2E). This indicates that it is possible to manipulate the bile acid feedback mechanism (mediated via tauro-beta-murocholic acid) in the host through gut expression of BSH. The data demonstrate for the first time that the effect of elevated BSH activity in the gut is to reduce total plasma bile acid levels, to reduce tauro-alpha and tauro-beta murocholic acid levels and to lower cyp7a1 expression. A role for the microbiota in modulating bile acid biosynthesis in both mice) and rats has been shown previously, however our study specifically demonstrates that bacterial bile salt hydrolase activity is central to this interplay between microbe and host.

Overall, the data indicate that the induction of in situ BSH activity in the model system significantly redirected the plasma bile acid signature (FIG. S1). PCA analysis showed detectable group differences using pareto-scaling in OPLS-DA. Here separation of the groups is easily visible and the noise levels are indicative of their degree of separation. They are 9% GF vs ConvR; 15% GF vs EC; 18% EC vs ECBSH1; 63% EC vs ECBSH2. Bile acid intensities identified as significantly different include increases in Taurine (3.8 fold), cholic acid (77 fold) and in TbMCA (407 fold) in GF and CONV-D comparisons. GF animals recolonized with E. coli alone showed substantial increases in the intensity of the following BAs; TbMCA (209 fold), cholic acid (50 fold) and b muricholic acid (22 fold). The presence of ECBSH1 reduced the intensity of Tauro-cholic acid (12 fold) and TbMCA (27 fold) in comparison with EC-colonized mice. As expected, we failed to detect significant levels of secondary or tertiary bile acids in gnotobiotic mice although these were abundant in CONV-D mice (FIG. 2F).

### Impact of Gastrointestinal Bile Salt Hydrolysis on Local and Systemic Gene Expression Patterns in the Host

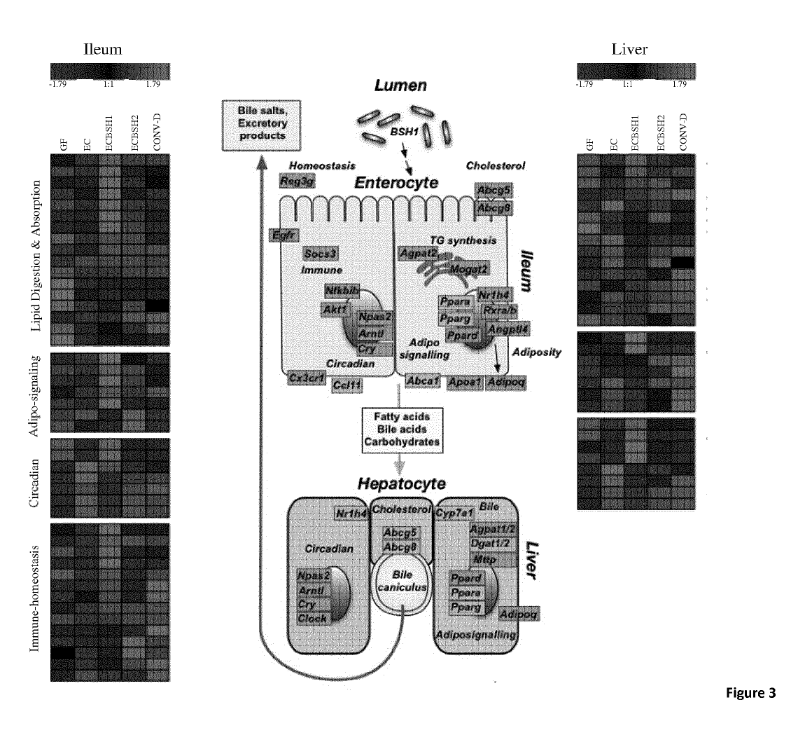

The expression patterns of over 23,000 genes in the liver and ileum in GF, monocolonised (EC, ECBSH1 or ECBSH2) and CONV-D mice were examined. Overall there were significant changes in host gene expression patterns induced by BSH1 and BSH2 relative to EC colonised mice, in both the ileum and the liver. Gene annotation and pathway mapping were employed using Subio software to examine the primary functional groups of host genes regulated through in situ expression of BSH enzymes in the host GI tract. Due to the potent activity of BSH1 in vitro and in vivo herein we focus primarily upon the influence of BSH1 expression in our system. However many of the loci influenced by BSH1 are also influenced by BSH2 activity (FIG. 3). The figure outlines selected genes in which BSH activity significantly modulated expression levels relative to the E. coli (EC) control. In the ileum BSH1 activity altered expression of loci associated with immune function, cholesterol transport and lipid transport and synthesis (FIG. 3). Gene expression was also significantly altered in the livers of mice following gastrointestinal colonisation by ECBSH1, with the regulation of major metabolic pathways involved in triglyceride biosynthesis, bile synthesis and fatty acid transport and synthesis. The major regulators of adipose tissue remodelling and peroxisome development, peroxisome proliferator-activated receptors (PPARs) were modulated by BSH in this system.

In addition BSH1 activity was a potent local trigger of the gene encoding the hormone adiponectin (adipoQ) as well as the gene encoding Angiopoietin-4 (also known as fasting induced adipose factor (FIAF)). Also note was the significant alteration of pathways regulated by circadian rhythm that have previously been implicated in energy metabolism and obesity (Costa et al., 2011). Genes encoding proteins with a known function in epithelial homeostasis and differentiation (EGFr, RegIIIg) were also strongly induced by BSH1 activity in our system. Gene expression profiles for a number of target genes were verified using qRT-PCR (FIG. S2). The data definitively demonstrate a conserved mechanism for molecular interaction between the microbiota and the host that is mediated by bacterial bile hydrolysis and which influences gene pathways involved in host cholesterol and lipid metabolism, immune function and circadian rhythm.

### Functional Consequences of Elevated Gastrointestinal BSH Activity in Conventionally Raised Mice

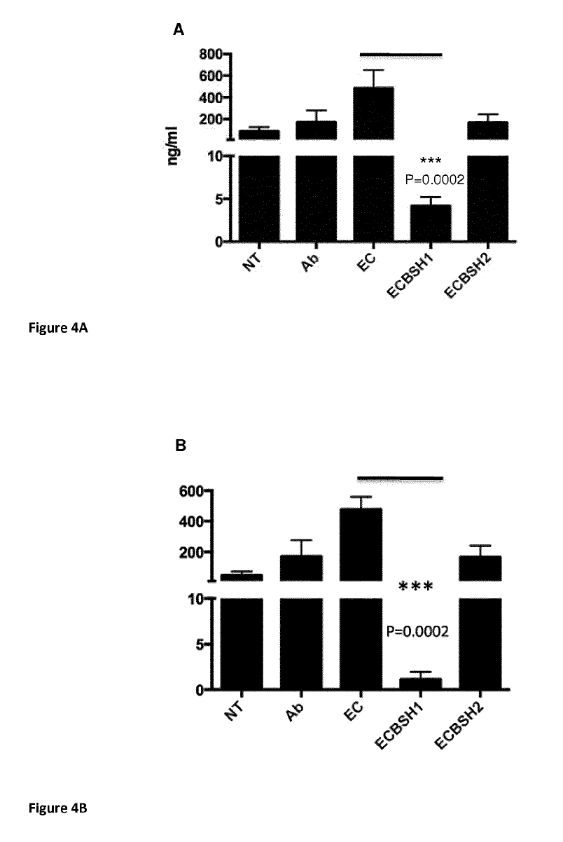

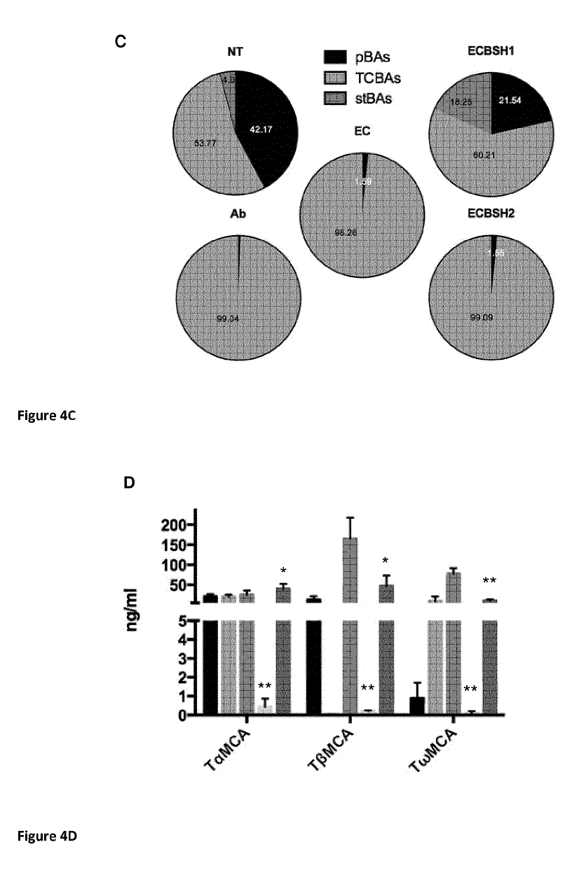

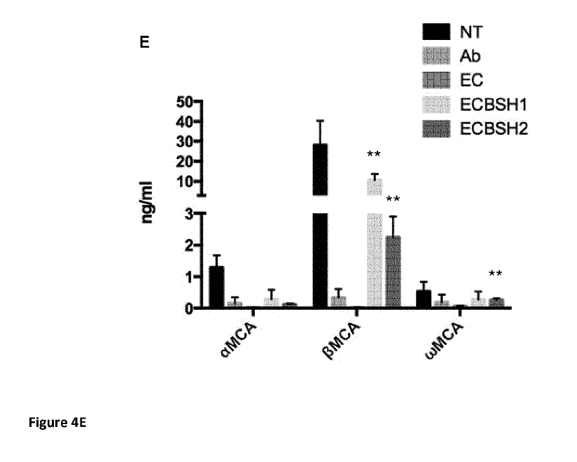

Given the influence of bacterial BSH on host energy pathways under controlled conditions in gnotobiotic mice, it was examined whether modulation of gastrointestinal BSH activity could form the basis of an intervention strategy for the control of host weight gain and metabolic processes in conventionally raised animals. In order to obtain consistent, high level expression of gastrointestinal BSH we again utilised the E. coli MG1655 gut colonisation model in which conventional streptomycin-treated mice were significantly colonised for over 70 days with strep^{R} E. coli alone (EC) or E. coli expressing BSH1 or BSH2 enzymes (FIG. S3). Expression of BSH1 in situ in the murine gut resulted in a significant increase in bile deconjugation activity resulting in a reduction in total plasma bile acids (FIG. 4A), a reduction in tauroconjugated bile acids in plasma (FIG. 4B) and a proportional increase in unconjugated primary bile acids (FIG. 4C). Gastrointestinal expression of BSH in conventional mice resulted in a dramatic reduction in plasma tauro-beta-murocholic acid (FIG. 4D) and a concomitant increase in levels of beta-murocholic acid (FIG. 4E) relative to EC colonised animals. Taken together the data show that it is possible to substantially manipulate bile acid profiles in conventionally raised mice through alteration of microbial BSH activity.

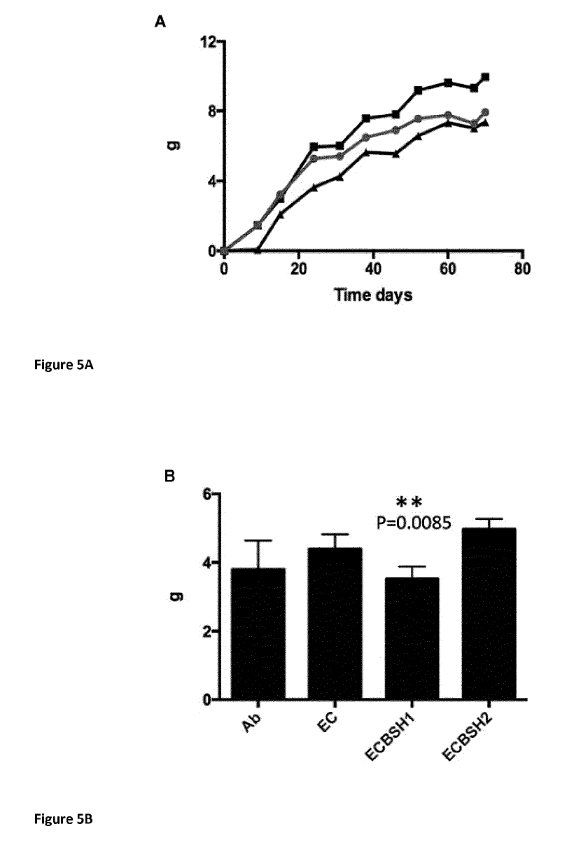

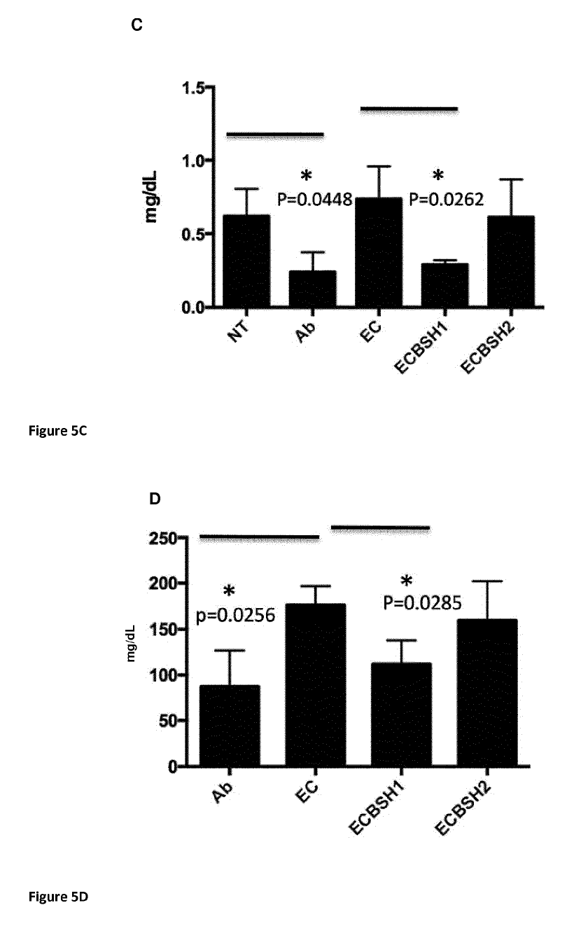

Colonisation of conventional mice by ECBSH1 resulted in significantly decreased weight gain (46% reduction) relative to mice colonised by E. coli alone (EC) in animals fed either a normal fat (FIG. 5A) or a high fat diet (HFD) (FIG. S4A). This was associated with reduced fat deposition in these animals (FIGS. 5B and S4B). BSH1 expression was also capable of lowering serum cholesterol (LDL cholesterol) and liver triglycerides relative to mice colonised by EC (FIGS. 5C and 5D). Similar results were seen in mice fed a HFD (FIGS. S4C and S4D). We noted that colonisation of mice with E. coli alone resulted in an increase in weight gain, supporting recent studies which link increases in body mass to increases in Proteobacteria, including E. coli. In our system BSH1 activity reversed this increase in weight gain. Significantly, we did not see an increase in systemic inflammation in our model (FIG. S5). Collectively the data show that BSH activity can be manipulated in the host gastrointestinal tract through a simple microbial intervention to moderate weight gain and cholesterol levels against the background of an existing microbiota.

### Effects of Elevated BSH Activity Upon Local and Systemic Transcriptional Patterns in the Host

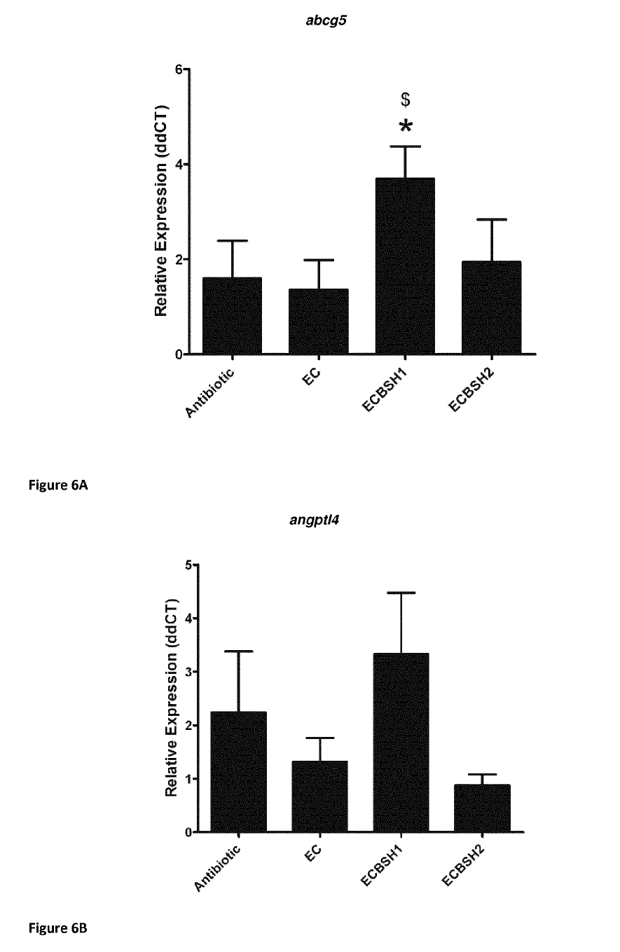

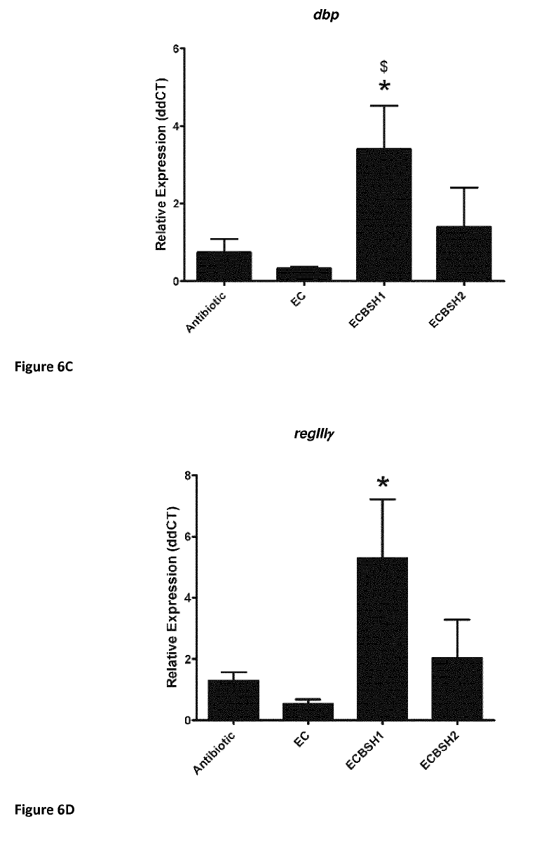

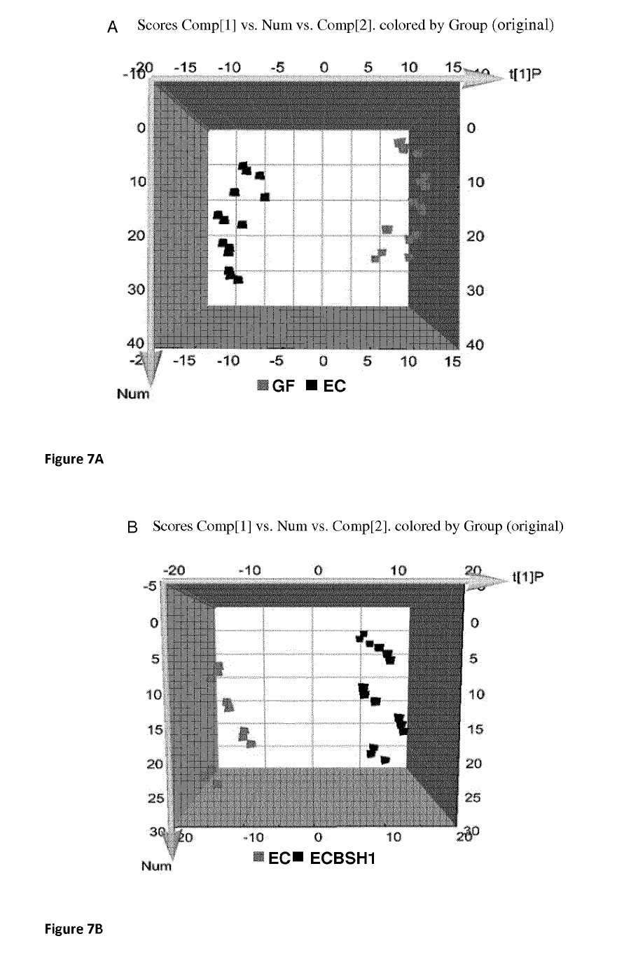

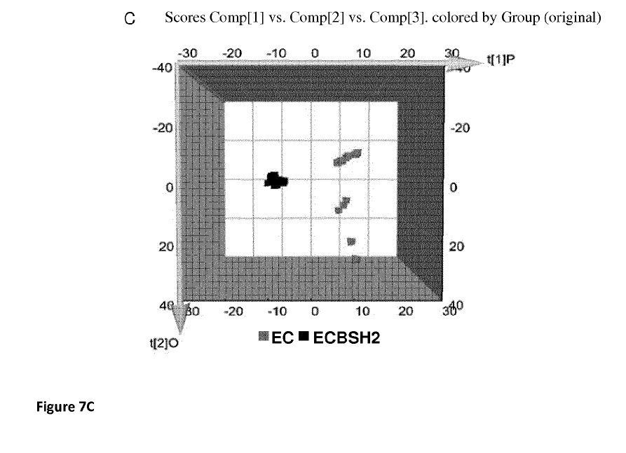

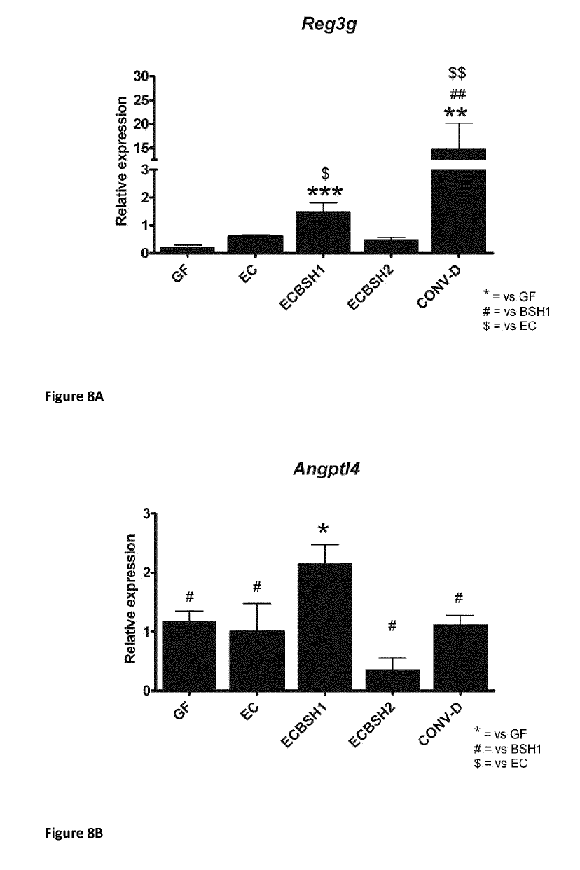

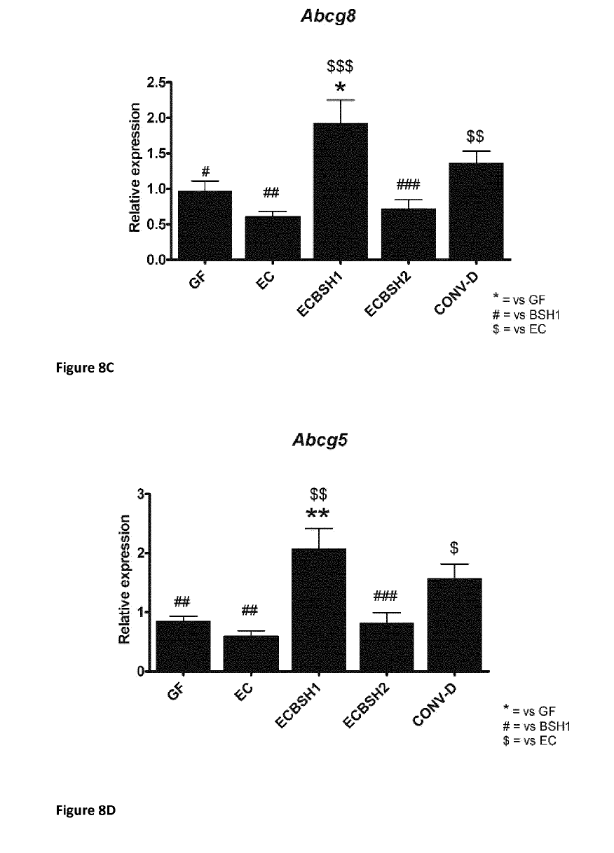

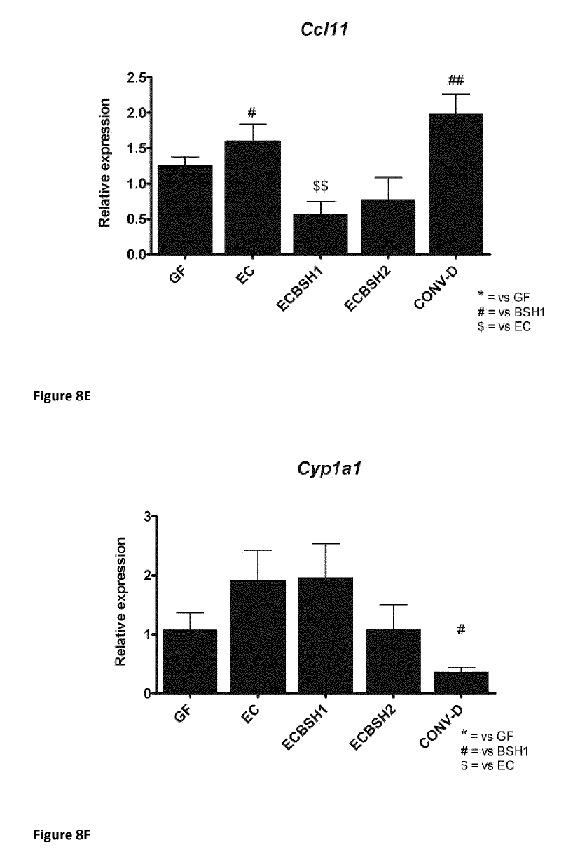

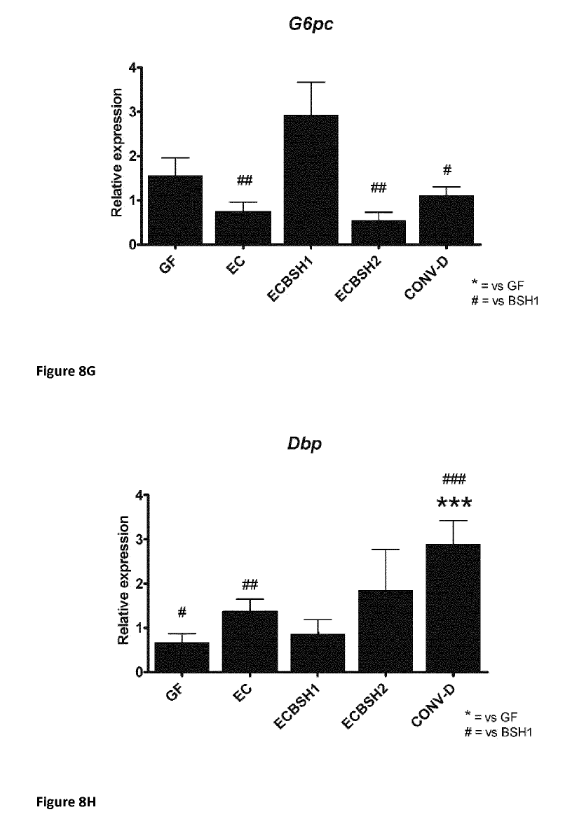

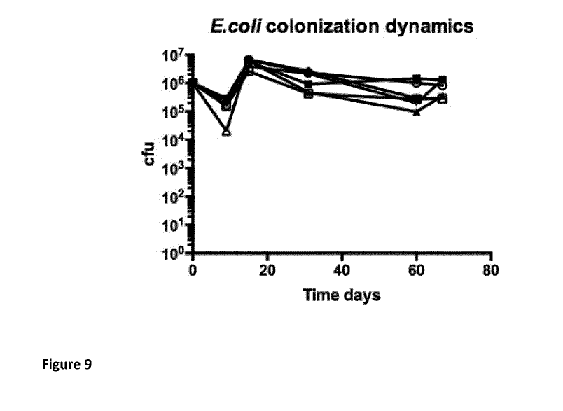

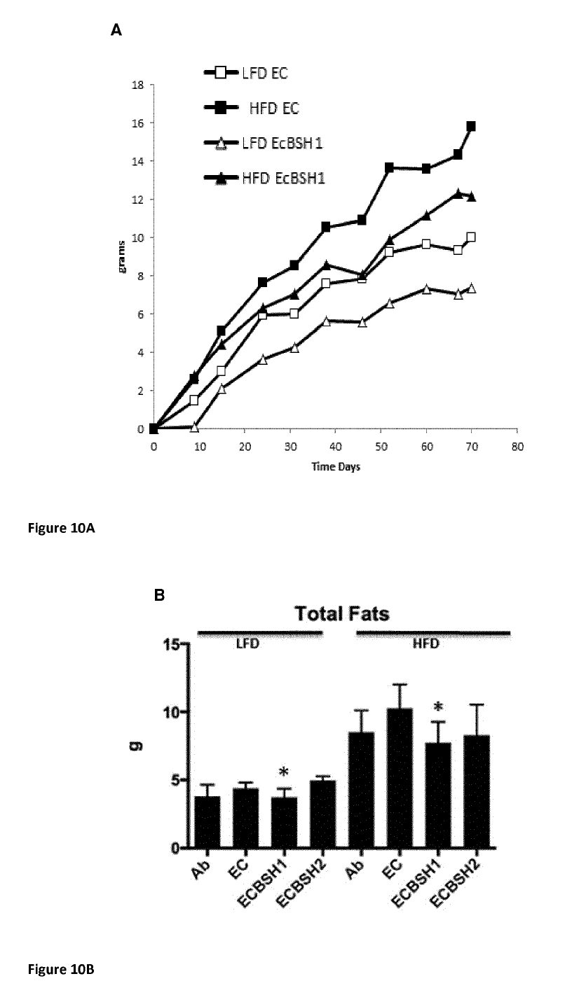

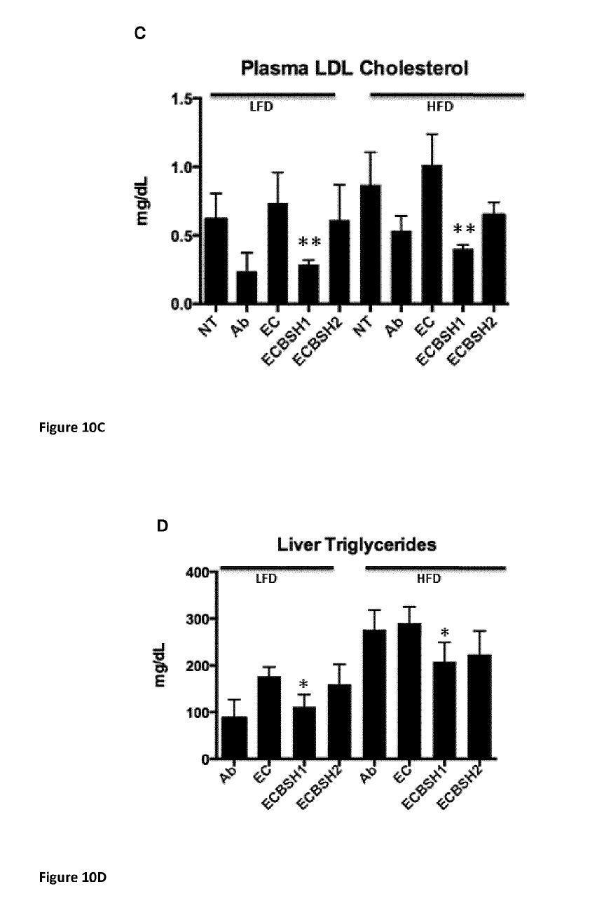

The Applicant has identified, using mono-colonised gnotobiotic mice, a number of host pathways that are clearly affected by gastrointestinal BSH activity (FIG. 3). Given the phenotypic changes in host physiology seen in conventionally raised animals, the gene expression profiles of a number of key genes in conventionally raised mice colonised by ECBSH1 or ECBSH2 were also examined (FIG. 6). The expression of these selected target genes was analysed using qRT-PCR. In particular, an increase in intestinal gene expression of abcg5/8 was detected in mice colonised by ECBSH1. BSH1 activity induced local expression of the angpt14 gene encoding FIAF, a lipoprotein lipase inhibitor that is known to be influenced by the microbiota. Gastrointestinal BSH1 activity also induced elevated expression of dbp a gene encoding a central regulator of circadian rhythm. BSH1 activity in conventional mice also induced ileal expression of regIIIg which encodes a secreted antibacterial lectin Levels of cdkn1a, a gene encoding a regulator of cell cycle (p21) were also elevated by BSH1 in conventionally raised mice.

### Comparison of Bile Hydrolase Activity of Isolates Using UPLC-MS:

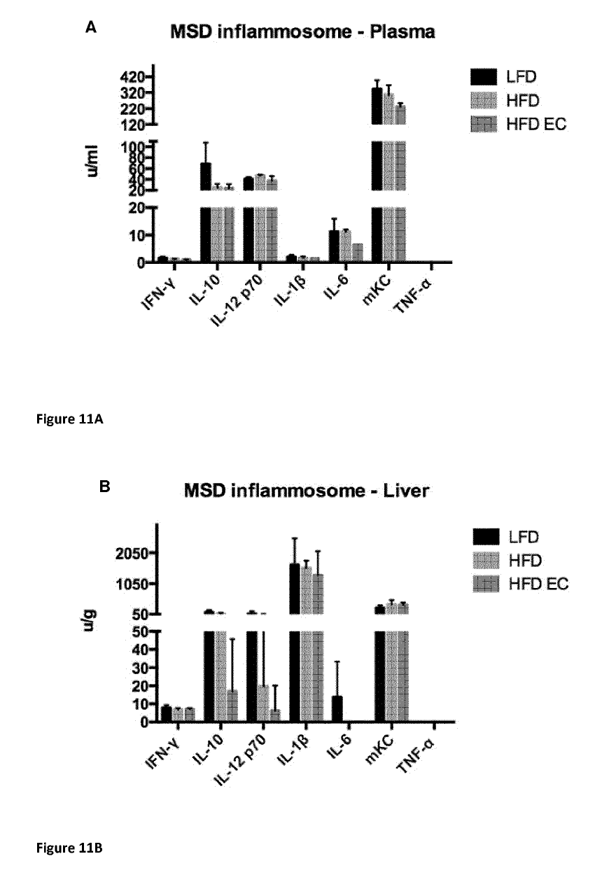

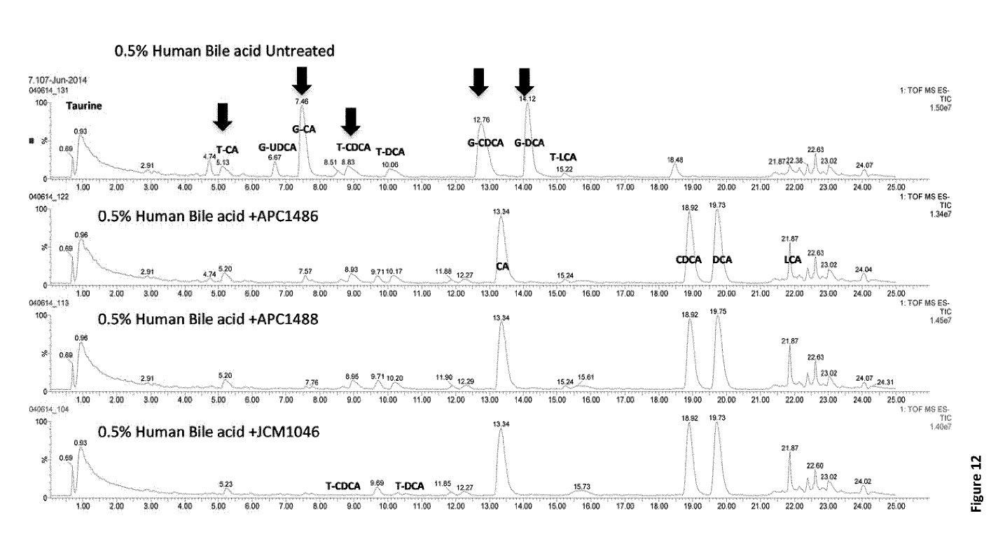

FIG. 12 shows the bile acid deconjugation effects of three strains of bacteria on human bile acid, strain APC1486 that expresses a BSH1 enzyme having 96% sequence identity with SEQUENCE ID NO: 1, strain APC1488 t expresses a BSH1 enzyme having 96% sequence identity with SEQUENCE ID NO: 1, and strain JCM1046 expresses a BSH1 enzyme having 100% sequence identity with SEQUENCE ID NO: 1.

The invention is limited to the embodiments hereinbefore described which may be varied in construction and detail without departing from the spirit of the invention.

APPENDIX
BSH1 SEQUENCES
SEQUENCE ID NO: 2 >gi|221062121|gb|FJ591081.1| Lactobacillus salivarius
strain JCM1046 megaplasmid bile salt hydrolase (bsh1) gene, complete
cds
ATGTGTACAGCAATTACTTTAAATGGTAATAGTAATTATTTTGGAAGAAATTTAGATTTGGATTTTTCATA
TGGCGAGCAGGTAATCATTACTCCGGCTGAGTATGAGTTTAAATTTAGAAAGGAAAAAGCTATAAAGAATC
ATAAATCATTGATAGGTGTTGGAATTGTCGCTAACGCTTACCCATTGTATTTTGATGCTATTAATGAGGAT
GGACTAGGAATGGCAGGATTGAATTTTCCTGGAAATGCATATTATAGCGATGCTTTAGAGAATGATAAAGA
TAATATTACGCCGTTCGAGTTTATTCCATGGATTCTGAGACAGTGTAGCGATGTTAATGAAGCAAGAAATT
TAGTTGAAAGAATAAATCTCATTAATCTTAGTTTTAGCGAACAATTACCTTTAGCAGGGTTACATTGGTTA
ATTGCAGATAGAGAAAAATCCATTGTAGTAGAAGTAACTAAATCTGGCGTACATATTTATGATAATCCAAT
TGGAGTATTGACTAATAATCCGGAATTTAATTATCAGATGTACAATCTGAATAAATATCGCAACTTATCTA
TCAGTACACCACAAAATACATTCTCAGATAGCGTGGATTTAAAAGTAGACGGTACCGGTTTTGGTGGTATT
GGCTTACCAGGCGATGTATCTCCCGAATCTCGTTTTGTGAGAGCTGCTTTTAGCAAGTTAAATTCAAGTAA
AGGGACGACCGTAGAAGAAGATATTACTCAGTTTTTTCATATACTAGGGACAGTAGAACAGATAAAGGGCG
TTAATAAGACAGAATCAGGAAAAGAAGAATATACTGTATATTCGAATTGTTATGATTTGGACAACAAGACG
TTATATTATACAACCTATGAAAATAGACAAATAGTAGCTGTTACTTTAAATGAAGATAAGAATGGTAATGG
GTTAATTGCATATCCATTTGAAAGAAAACAAGTAATAAATAAGTTGAATTAA
SEQUENCE ID NO: 3 >APC1484.seq-ID: Pig A1 BSH-BSH1 Forward on
2014 May 28-21:57:35 automatically edited with PhredPhrap, start with
base no.: 1 Internal Params: Windowsize: 20, Goodqual: 19, Badqual:
10, Minseqlength: 50, nbadelimit: 1
TTGGaGanCnTagatTTGgaCTTTncataTGGCGAGCAGGTAATCATTACTCCGGCTGagtATGAATtTAA
ATTTAGAAAGGAAAAGGCTATAAAGAATCATAAATCATTAATAGGTGTTGGAATTGTCGCTGACGATTACC
CATTGTATTTTGATGCtaTTAATGAGGATGGACTAGGAATGGCAGGATTGAATTTTCCTGGAAATGCATAT
TATAGCGATTTTTTAGAGAATGACAAAGATAATATTACGCCATTTGAGTTTATTCCATGGATTCTGGGACA
GTgTAGCGATGTTAATGAAGCAAGAAATTTAGTTGAAAGAATAAATCTCATTAATCTTAGTTTTAGCGAAC
AATTACCTTTAGCAGGGTTACATTGGTTAATTGCAGATAGAGAAAAATCTATTGTAGTAGAAGTAACTAAA
TCTGGCGTACATATTTATGATAATCCAATTGGAGTATTGACTAATAATCCGGAATTTAATTATCAGATGTA
TAATCTGAATAAATATCGCAACTTATCTATCAGTACACCACAAAATACATTCTCAGATAGCGTGGATTTAA
AGGTAGACGGTACTGGTTTTGGCGGTATTGGCTTACCAGGCGATGCATCTCCCGAATCTCGTTTTGTGAGA
GCTGCTTTTAGCAAGTTAAATTCAAGTAAAGGGACGACCGTAGAAGAAGATATTACTCAGTTTTTCCATAT
ACTAGGGACAGTAGAACAGATAAAGGGCGTTAATAAGACAGAATCAGGAAAAGAAGAATATACTGTATATT
CGAATTGCTATGATTTGGACAACAnAACGTTATATTATACAACCTATGAAnATAGACAAATAGTGTCTGTT
ACTTtAnATAAAGATAAGAATGGTAATAAGTTAGTCGTATATCCATTTGAaaGanAACAAGTAATAAAtag
ntTGAATTAAt
SEQUENCE ID NO: 5 >APC1485.seq-ID: Pig A2 BSH-BSH1 Forward on
2014 May 28-21:57:35 automatically edited with PhredPhrap, start with
base no.: 10 Internal Params: Windowsize: 20, Goodqual: 19, Badqual:
10, Minseqlength: 50, nbadelimit: 1
CnTagatTTGgatTTTtcaTaTGGCGAGCaGGTAATCaTTACTCCGGCTGAGtATGAGTTTAaATTTaGAA
AGGAAAAGGCtaTAAAGAATCATAAATCATTAATAGGTGTTGGAATTGTCGCtAACGATTACCCAtTgTAT
TTTGATGCtatTAATGAGGATGGATTAGGAATGGCAGGATTGAATTTTCCTGGAAATGCATATTATAGCGA
TGCTTTaGAGAATGACAAAgaTAATATTACACCGTTCGAGTTTATTCCATgGATtcTGGgaCagtgtaGCG
AtgtTAATGAAGCAAGAAATTTAGTTGAAAGAATAAATCTCATTAATCTTAgtTTTAGCGAACAATTACCT
TTAGCAGGATTACATTggTTAATTGCTGATAGAGAAAAATCCATTGTAGTAGAAgtaACTAAATCTGGCGT
ACATATTTATGATAATCCAATTGGAGTATTGACTAATAATCCGGAATTTAATTATCAGATGTACAATCTGA
ATAAATATCGCAACTTATCTATCAGTACACCACAAAATACATTCTCAGATAGCGtgGATTTAAAGGTAGAC
GGTACTGGTTTTGGTGGTATTGGCTTACCAGGCGACGTATCTCCCGAATCTCGTTTTGTGAGAGTTGCTTT
TAGCAAGTTAAATTCaaATAAAGGAACGACCGTagAAGAAGATATTACTCAGTTTTTCCATATACTaggGA
CAGTagaACAGATAAAGGGTGTTAATAAGaCAGAATCAGGAAAAGAAGAATATACTGTATATTCGAAttGC
TATaaTtngGACAACnnaACGttaTattATACAACCTATgaAaATagAccaATAGTGTcTgTTACTttana
TaaaGataAGAATGgtAATAAGTTAGTCGTATATCCATTTGnnagAAAACAAGTAanaaaTaggt
SEQUENCE ID NO: 7 >APC1486.seq-ID: Pig A3 BSH-BSH1 Forward on
2014 May 28-21:57:35 automatically edited with PhredPhrap, start with
base no.: 1 Internal Params: Windowsize: 20, Goodqual: 19, Badqual:
10, Minseqlength: 50, nbadelimit: 1
TTtGGaGnaCnTagatTTGganTTTtCaTatGGCGAGCaGGTAATCattACTCCGGCTGagtATGAGTTTa
AATTTaGAAAGGAAAAGGCTATAAAGAATCATAAATCATTAATAGGTGTTGGAATTGTCGCnnaCGATTAC
CCAtTGTATTTTGATGCTATTAATGAGGATGGattAGGAATGGCAGGATTGAATTTTCCTGGAAATGCATA
TTATAGCGAtnntTTAGAGAATGACAAAGATAATATTACACCGTtcgAGTTTATTCCATgGATTCTGGgaC
AgtgtaGCGAtgTTAATGAAGCAAGAAATTTAGTTGAAAGAATAAATCTCATTAATCTTAGTTTTAGCGAA
CAATTACCTTTAGCAGGATTACATTGGTTAATTGCTGATAGAGAAAAATcnaTTGTAGTAGAAGTAACTAA
ATCTGGCGTACATATTTATGATAATCCAATTGGAGTATTGACTAATAATCCGGAATTTAATTATCAGATGT
ACAATCTGAATAAATATCGCAACTTATCTATCAGTACACCACAAAATACATTCTCAGATAGCGTGGATTTA
AAGGTAGACGGTACTGGTTTTGgtgGTATTGGCTTACCAGGCGacGtATCTCCCGAATCTCGTTTTGTGAG
AgntGCTTTTAGCAAGTTAAATTCAaatAAAGGAACGACCGTAGAAGAAGATATTACTCAGTTTTTCCATA
TACTAGGGACAGTAGAACAGATAAAGGgngTTAATAAGaCAGAATCAGGAAAAGAAGAATATACTGTATAT
TCGAATTGCTAtgaTTTGGACAACanaACGTTATATTATACAACCTATGAAAATAGAcaaATAGTGTCTGT
TACTTTAAATAAAGATAAGAATGGTAATAAGTTAGTCGTATATCCATTTGAAAGAAAACAAGTAATAAATa
gnnt
SEQUENCE ID NO: 9 >APC1487.seq-ID: Pig A4 BSH-BSH1 Forward on
2014 May 28-21:57:35 automatically edited with PhredPhrap, start with
base no.: 31 Internal Params: Windowsize: 20, Goodqual: 19, Badqual:
10, Minseqlength: 50, nbadelimit: 1
ttCntaTGgCGAGCaGGtAatcattACTCCGGCtgagTATGAGTttAaaTtTaGAAAGGAAAAGGCtaTAA
AGAATCATAAATCAttaataGgtGTtGGAATtgTCGCTAACGAttaCCCAttgtATTTTGATGCtAttaAT
GAGGaTGGATTAGGAATGGCAGGATTGAATTTTCCTgGAAATGCATATTATAGCGATGCTTTaGAGAATGA
CAAAGaTAATATTACACCGTTCGAGTTTATTCCATgGaTtctGGgacAgtgtaGCGATGtTaATGAAGCAA
GAAATTTagTTGAAAGAATAAATCTCATTAATCTTAGtTTTAGCGAACAATTACCTTTAGCAGGATTACAT
TGGTTAATTGCTGATAGAGAAAAATCCATTGTAGTAGAAgnaACTAAATCTGGCGTACATATTTATGATAA
TCCAATTGGAGTATTGACTAATAATCCGGAATTTAATTATCAGATGTACAATCTGAATAAATATCGCAACT
TATCTATCAGTACACCACAAAATACATTCTCAGATAGCGTGGATTTAAAGGTAGACGGTACTGGTTTTGGT
GGTATTGGCTTACCAGGCgACGTATCTCCCGAATCTCGTTTTGTGAGAGTTGCTTTTAGCAAGTTAAATTC
AAATAAAGGAACGACCGTAGAAGAAGATATTACTCAGTTTTTCCATATACTaggGACAGTanaACAGATAA
AGGGTGTTAATAAGACAGAATCagGAaAAGAAGAATATACTgnaTATTCgAATTGCTATaATttGGACAAC
aaAACGttaTATtATACAACCTATGAAaATnnACAaatAGTGTCTgTTActttanataaaGATAaGAATGg
taATAAgttAgtcgT
SEQUENCE ID NO: 11 >APC1488.seq-ID: Pig AS BSH-BSH1 Forward on
2014 May 28-21:57:35 automatically edited with PhredPhrap, start with
base no.: 64 Internal Params: Windowsize: 20, Goodqual: 19, Badqual:
10, Minseqlength: 50, nbadelimit: 1
GCTGAGtATGAATttaAATTTaGAAAGGAAAAGGCTATAAAGAATCATAAATCATTAATAGGTGTTGGAAT
TGTCGctGACGATTACCCATTGTATTTTGATGCtATTAATGAGGATGGACTAGGAATGGCAGGATTGAATT
TTCCTGGAAATGCATATTATAGCGATTTTTTAGAGAATGACAAAGaTAATATTACGCCATTTGAGTTTATT
CCATGGATTCTGGGACAGtgtAGCGaTgTTAATGAAGCAAGAAATTTAGTTGAAAGAATAAATCTCATTAA
TCTTAGTTTTAGCGAACAATTACCTTTAGCAGGGTTACATTGGTTAATTGCAGATAGAGAAAAATCTATTG
TAGTAGAAGTAACTAAATCTGGCGTACATATTTATGATAATCCAATTGGAGTATTGACTAATAATCCGGAA
TTTAATTATCAGATGTATAATCTGAATAAATATCGCAACTTATCTATCAGTACACCACAAAATACATTCTC
AGATAGCGTGGATTTAAAGGTAGACGGTACTGGTTTTGGCGGTATTGGCTTACCAGGCGATGCATCTCCCG
AATCTCGTTTTGTGAGAGCTGCTTTTAGCAAGTTAAATTCAAGTAAAGGGACGACCGTAGAAGAAGATATT
ACTCAGTTTTTCCATATACTAGGGACAGTAGAACAGATAAAGGGCGTTAATAAGACAGAATCAGGAAAAGA
AGAATATACTGTATATTCGAATTGCTATGATTTGGACAACAAAACGTTATATTATACAACCTATGAAAATA
GACAAATAGTGTCTGTTACTTtanATAAAGATAAGAATGGTAATAAGTTAGTCGTATATCCATTTGAAAGA
nAACAAGTAATAAATAagttTGAATTAAAAa
SEQUENCE ID NO: 13 >APC1489.seq-ID: Pig A6 BSH-BSH1 Forward on
2014 May 28-21:57:36 automatically edited with PhredPhrap, start with
base no.: 1 Internal Params: Windowsize: 20, Goodqual: 19, Badqual:
10, Minseqlength: 50, nbadelimit: 1
TTGGaGnaCtTaGatTTGGaCTTTnnataTGGCGAGCAGGTAATCATTACTCCGGCTGAGTATGAATTTAA
ATTTAGAAAGGAAAAGGCTATAAAGAATCATAAATCATTAATAGGTGTTGGAATTGTCGCTGACGATTACC
CATtgTATTTTGATGCTATTAATGAGGATGGACTAGGAATGGCAGGATTGAATTTTCCTGGAAATGCATAT
TATAGCGATTTTTTAGAGAATGACAAAGATAATATTACGCCATTTGAGTTTATTCCATGGATTCTGGGACA
GTGTAGCGATGTTAATGAAGCAAGAAATTTAGTTGAAAGAATAAATCTCATTAATCTTAGTTTTAGCGAAC
AATTACCTTTAGCAGGGTTACATTGGTTAATTGCAGATAGAGAAAAATCTATTGTAGTAGAAGTAACTAAA
TCTGGCGTACATATTTATGATAATCCAATTGGAGTATTGACTAATAATCCGGAATTTAATTATCAGATGTA
TAATCTGAATAAATATCGCAACTTATCTATCAGTACACCACAAAATACATTCTCAGATAGCGTGGATTTAA
AGGTAGACGGTACTGGTTTTGGCGGTATTGGCTTACCAGGCGATGCATCTCCCGAATCTCGTTTTGTGAGA
GCTGCTTTTAGCAAGTTAAATTCAAGTAAAGGGACGACCGTAGAAGAAGATATTACTCAGTTTTTCCATAT
ACTAGGGACAGTAGAACAGATAAAGGGCGTTAATAAGACAGAATCAGGAAAAGAAGAATATACTGTATATT
CGAATTGCTATGATTTGGACAACAAAACGTTATATTATACAACCTATGAAAATAGACAAATAGTGTCTGTT
ACTTTAnATAAAGATAAGAATGGTAATAAGTTAGTCGTATATCCATTTGAAAGAAAACAAGTAataAATan
gntt
SEQUENCE ID NO: 15 >APC1490.seq-ID: Pig A7 BSH-BSH1 Forward on
2014 May 28-21:57:36 automatically edited with PhredPhrap, start with
base no.: 10 Internal Params: Windowsize: 20, Goodqual: 19, Badqual:
10, Minseqlength: 50, nbadelimit: 1
CnTagatTTGGaCTTTtcaTaTGGCGAGCAGGTAATCATTACTCCGGCTGAGtATGAATTTAAATTTAGAA
AGGAAAAGGCTATAAAGAATCATAAATCATTAATAGGTGTTGGAATTGTCGCTGACGATTACCCATtgTAT
TTTGATGCTATTAATGAGGATGGACTAGGAATGGCAGGATTGAATTTTCCTGGAAATGCATATTATAGCGA
TTTTTTAGAGAATGACAAAGATAATATTACGCCATTTGAGTTTATTCCATGGATTCTGGGACAGTgTAGCG
ATGTTAATGAAGCAAGAAATTTAGTTGAAAGAATAAATCTCATTAATCTTAGTTTTAGCGAACAATTACCT
TTAGCAGGGTTACATTGGTTAATTGCAGATAGAGAAAAATCTATTGTAGTAGAAGTAACTAAATCTGGCGT
ACATATTTATGATAATCCAATTGGAGTATTGACTAATAATCCGGAATTTAATTATCAGATGTATAATCTGA
ATAAATATCGCAACTTATCTATCAGTACACCACAAAATACATTCTCAGATAGCGTGGATTTAAAGGTAGAC
GGTACTGGTTTTGGCGGTATTGGCTTACCAGGCGATGCATCTCCCGAATCTCGTTTTGTGAGAGCTGCTTT
TAGCAAGTTAAATTCAAGTAAAGGGACGACCGTAGAAGAAGATATTACTCAGTTTTTCCATATACTAGGGA
CAGTAGAACAGATAAAGGGCGTTAATAAGACAGAATCAGGAAAAGAAGAATATACTGTATATTCGAATTGC
TATGATTTGGACAACAAAACGTTATATTATACAACCTATGAAAATAGACAAATAGTGTCTGTTACTTTAAA
TAAAGATAAGAATGGTAATAAGTTAGTCGTATATCCATTTGAnAGAnAACAAGTAATAAATAggTtTGAAT
TAAaaa
SEQUENCE ID NO: 17 >APC1491.seq-ID: Pig A8 BSH-BSH1 Forward on
2014 May 28-21:57:36 automatically edited with PhredPhrap, start with
base no.: 9 Internal Params: Windowsize: 20, Goodqual: 19, Badqual:
10, Minseqlength: 50, nbadelimit: 1
CnTaGaTTtGgaCTTTtcaTaTGGCGAGCaGGTAATCATTACTCCGGCTGAGtATGAATtTAAATTTAGAA
AGGAAAAGGCtATAAAGAATCATAAATCATTAATAGGTGTTGGAATTGTCGCTGACGATTACCCAtTGTAT
TTTGATGCtatTAATGAGGaTGGACtAGGAATGGCAgGATTGAATTTTCCTGGAAATGCAtaTTATAGCGA
TTTTTTAGAGAATGACAAAGaTAATATTACGCCATTtgAGTTTATTCCATgGATTCTGGGaCagtgtaGCG
ATGTTAATGAAGCAAGAAATTTAGTTGAAAGAATAAATCTCATTAATCTTAgtTTTAGCGAACAATTACCT
TTAGCAGGGTTACATTGGTTAATTGCAGATAGAGAAAAATCTATTGTAGTAGAAGTAACTAAATCTGGCGT
ACATATTTATGATAATCCAATTGGAGTATTGACTAATAATCCGGAATTTAATTATCAGATGTATAATCTGA
ATAAATATCGCAACTTATCTATCAGTACACCACAAAATACATTCTCAGATAGCGTGGATTTAAAGGTAGAC
GGTACTGGTTTTGGCGGTATTGGCTTACCagGCGATGCATCTCCCGAATCTCGTTTTGTGAGAGCTGCTTT
TAGCAAGTTAAATTCAAGTAAAGGgaCGACCGTAgaAGAAGATATTACTCAGTTTTTCCATATACTAGGGA
CAGTAGAACAGATAAAGGGCGTTAATAAGncaGAATCAGGAAAAGAAGAATATACTGTATATTCGaATTGC
TATGATTTGgacAACAAAACGttATATTATACAACCTATGAAAATAGACAAATAGTGTCTGTTACTttnna
taaAGATAAgaATGGTAAtaaGTTAGTCGtATATCCATTTGAAAGAAAAcAAGTAAtnaaTAg
SEQUENCE ID NO: 19 >APC1492.seq-ID: Pig A9 BSH-BSH1 Forward on
2014 May 28-21:57:36 automatically edited with PhredPhrap, start with
base no.: 14 Internal Params: Windowsize: 20, Goodqual: 19, Badqual:
10, Minseqlength: 50, nbadelimit: 1
gatTTGgactTTtcaTaTGGCGAGCAGGTAATCATTACTCcgGCTgagtATGAATTtaaATttaGAAAGGA
AAAGGCtaTAAAGAATCATAAATcatTAATAGgtgTTGGAATTGTCGCtGACGATTACCCAttgtATTTTG
ATGCtatTAATGAGGaTGGACTAGGAATGGCAGGATTGAATTTTCCTGgaAATGCATATTATAGCGATTTT
TTAGAGAATgaCAAAGATAATATTACGCCATTTGAGTTTATTCCATgGATTCTGGGACagtgtaGCGAtgt
TAATGAAGCAAGAAATTTAGTTGAAAGAATAAATCtCATTAATCTTAGTTTTAGCGAACAATTACCTTTAG
CAGGGTTACATTGGTTAATTGCAGATAGAGAAAAATCTATTGTAGTAGAAGTAACTAAATCTGGCGTACAT
ATTTATGATAATCCAATTGGAGTATTGACTAATAATCCGGAATTTAATTATCAGATGTATAATCTGAATAA
ATATCGCAACTTATCTATCAGTACACCACAAAATACATTCTCAGATAGCGTGGATTTAAAGGTAGACGGTA
CTGGTTTTGGCGGTATTGGCTTACCAGGCGATGCATCTCCCGAATCTCgttTTGTGAGAGCTGCTTTTAGC
AAGTTAAATTCAAGTAAAGGGACGACCGTAGAAGAAGATATTACTCAGTTTTTCCATATACTAGGGACAGT
anaACAGATAAAGGGCGTTAAtaAGaCAGAATCAGGAAAAGAAGAATATACTGTATATTCGAATTGCTATG
ATTTggaCAACAAAACgttATATTATaCAACCtaTGAAAATAGACAaATAGTGTCTGTTAcTTTAaatnaA
GATAAGAATGGTAATAAgntAGTCGTATATCCATTTgAAAGAAAACAAGTAAtaa
SEQUENCE ID NO: 21 >APC1493.seq-ID: Pig A10 BSH-BSH1 Forward on
2014 May 28-21:57:36 automatically edited with PhredPhrap, start with
base no.: 1 Internal Params: Windowsize: 20, Goodqual: 19, Badqual:
10, Minseqlength: 50, nbadelimit: 1
TTGGaGnaCnTagatTTGgaCTTTncanaTGGCGAGCagGTAATCatTACTCcgGCTGAGtATGAATtTaA
ATTTAGAAAGGAAAAGGCTATAAAGAATCATAAATCATTAATAGGTGTTGGAATTGTCGCTGACGATTACC
CAtTGTATTTTGATGCtatTAATGAGGATGGACtaGGAATGGCAGGATTGAATTTTCCTGGAAATGCATAT
TATAGCGATTTTTTAGAGAATGACAAAGaTAATATTACGCCATTTgAGTTTATTCCATgGATTCTGGgaCa
gngtaGCGATGTTAATGAAGCAAGAAATTTaGTTGAAAGAATAAATCTCATTAATCTTaGTTTTAGCGAAC
AATTACCTTTAGCAGGGTTACATTgGTTAATTGCAGATAGAGAAAAATCTATTGTAGTAGAAGTAACTAAA
TCTGGCGTACATATTTATGATAATCCAATTGGAGTATTGACTAATAATCCGGAATTTAATTATCAGATGTA
TAATCTGAATAAATATCgCAACTTATCTATCAGTACACCACAAAATACATTCTCAGATAGCGTGGATTTAA
AGGTAGACGGTACTGGTTTTGGCGGTATTggcTTACCagGCGATGCAtctCCCGAATCTCGTTTTGTGAGA
GCTGCTTTTAGCAAGTTAAATTCAAGTAAAGGgaCGACCGTagaAGAAGATATTACTCAgntTTTCCATAT
ACTAGGGACAGTagaACAGATAAAGGGCGTTAATAAGannGAATcagGAAAAgAagAATATACTGTATATT
CGaATTGCTATGATTTGGACAACaAAACGTTATATTATACAACCTATGAaaaTAGAcnnATAGTGTCTGTT
ACtnnnnaTaaaGATAAGAATGGTAAtaagtTAGTCGTATATCCATTTGAAagaAAACAAGTAATAAATAg
GTt
SEQUENCE ID NO: 23 >APC1494.seq-ID: Pig B1 BSH-BSH1 Forward on
2014 May 28-21:57:36 automatically edited with PhredPhrap, start with
base no.: 1 Internal Params: Windowsize: 20, Goodqual: 19, Badqual:
10, Minseqlength: 50, nbadelimit: 1
gaTGGaGaaCnTagatTTGGaCTTTncanaTGGCGAGCaggTAATCATTACTCcgGCtgaGtATGAATTTA
aaTTTaGAAAGGAAAAGGCtataAAGAATCATAAATCATTAATAGGTGTTGGAATTGTCGCtgACGATTAC
CCATtGTATTTTGATGCTATTAATGAGGaTgGACTAGGAATGGCAGGATTGAATTTTCCTgGAAATGCATA
TTATAGCGATTTTTTAgaGAATgACAAAgatAATATTACGCCATTtGAGTTTATTCcatgGATtctGGgaC
agngnaGCGatgtTAATGAAGCAAGAAATTtagtTGAAAGAATAAATCTCATTAATCTTagttTTAGCGAA
CAATTACCTTTAGCAGGGTTACATTggTTAATTGCAGATAGAGAAAAATCTATTGTAGTAGAAGTAACTAA
ATCTGGCGTACATATTTATGATAATCCAATTGGAGTATTGACTAATAATCCGGAATTTAATTATCAGATGT
ATAATCTGAATAAATATCgcAACTTATCTATCAGTACACCACAAAATACATTCTCAGATAGCGTGGATTTA
AAGGTAGACGGTaCTgGtTTTGGCGGTATTggcTTACCAggcgATGCatctCCCgaATCTCGtttTGTGAG
AGCTGCTtTTAGCAAGTTAAATTCAAGTAAAGggaCGACCGTanaagnaGATATTACTCAgntTttCCATA
TACTAGGGACAGTnnaACAGATAAAGGGCGTTAAtaaGannGAATcngGAAAAGAAGAATATACTGTATAT
TCgaATTGCTATGATTTGgACAAcaAAACGttATATTATACAACCTATGAaaaTAGAcaaATAGTGTctgT
TACtnnnnntnangATAAGaATgntAATaagtTAGTCGtATATCCATTTGaaAGAAaACAAGTAATAAAta
gnnt
SEQUENCE ID NO: 25 >APC1495.seq-ID: Pig B2 BSH-BSH1 Forward on
2014 May 28-21:57:36 automatically edited with PhredPhrap, start with
base no.: 1 Internal Params: Windowsize: 20, Goodqual: 19, Badqual:
10, Minseqlength: 50, nbadelimit: 1
TTtGGaGanCntaGatTTGgactTTnnataTGGCGAGCAGGTAATCATTACTCcgGCTGagtATGAATtta
AATTTAGAAAGGAAAAGGCTATAAAGAATCATAAATCATTAATAGGTGTTGGAATTGTCGCTGACGATTAC
CCATtgTATTTTGATGCTATTAATGAGGATGGACTAGGAATGGCAGGATTGAATTTTCCTGGAAATGCATA
TTATAGCGATTTTTTAGAGAATGACAAAGATAATATTACGCCATTTGAGTTTATTCCATGGATTCTGGGAC
AGTgTAGCGATGTTAATGAAGCAAGAAATTTAGTTGAAAGAATAAATCTCATTAATCTTAGTTTTAGCGAA
CAATTACCTTTAGCAGGGTTACATTGGTTAATTGCAGATAGAGAAAAATCTATTGTAGTAGAAGTAACTAA
ATCTGGCGTACATATTTATGATAATCCAATTGGAGTATTGACTAATAATCCGGAATTTAATTATCAGATGT
ATAATCTGAATAAATATCGCAACTTATCTATCAGTACACCACAAAATACATTCTCAGATAGCGTGGATTTA
AAGGTAGACGGTACTGGTTTTGGCGGTATTGGCTTACCAGGCGATGCATCTCCCGAATCTCGTTTTGTGAG
AGCTGCTTTTAGCAAGTTAAATTCAAGTAAAGGGACGACCGTAGAAGAAGATATTACTCAGTTTTTCCATA
TACTAGGGACAGTAGAACAGATAAAGGGCGTTAATAAGaCAGAATCAGGAAAAGAAGAATATACTGTATAT
TCGAATTGCTATGATTTGGACAACAAAACGTTATATTATACAACCTATGAAAATAGACAAATAGTGTCTGT
TACTTTAnATAAAGATAAGAATGGTAATAAGTTAGTCGTATATCCATTTGAnAGAAAACAAGTAATAAATA
agttTGAATTAAaa
SEQUENCE ID NO: 27 >APC1496.seq-ID: Pig B3 BSH-BSH1 Forward on
2014 May 28-21:57:36 automatically edited with PhredPhrap, start with
base no.: 30 Internal Params: Windowsize: 20, Goodqual: 19, Badqual:
10, Minseqlength: 50, nbadelimit: 1
ttnGtatGGCGAGcnggTAATCATTACTCCGGctgagtATGAGTTtAaaTttaGAAAGGAAAAGGCtatAA
AGAATCATAAATCATTAataGgtgTtgGAATtgTCGctAACGATTACCCATtgtATTTTGATGCTATTAAT
GAGGATGGATTAGGAATGGCAGGATTGAATTTTCCTGGAAATGCATATTATAGCGATGCTTTagagAATGA
CAAAGATAATATTACACCGtTcGagTTTATTCCATGGATtctGGgACagngnaGCGATGTTAATGAAGCAA
GAAATTTantTGAAAGAATAAATCTCATTAATCTTAGTTTTAGCGAACAATTACCTTTAGCAGGATTACAT
TGGTTAATTGCTGATAGAGAAAAATCCATTGTAGTAGAAgtAACTAAATCTGGCGTACATATTTATGATAA
TCCAATTGGAGTATTGACTAATAATCCGGAATTTAATTATCAGATGTACAATCTGAATAAATATCGCAACT
TATCTATCAGTACACCACAAAATACATTCTCAGATAGCGTGGATTTAAAGGTAGACGGTACTGGTTTTGGT
GGTATTGGCTTACCAGGCGACGTATCTCCCGAATCTCGTTTTGTGAGAGTTGCTTTTAGCAAgntAAATTC
AAATAAAGGAACGACCGTAGAAGAAGATATTACTCAGTTTTTCCATATACTAGGGACAGTAGAACAGaTAA
AGGGTGTTAaTAAGACAGAATCAGGaAAAGAAGAATATACTgtaTATTCGAATTGCTATAATTTGGACAAC
AAAACGTTATATTATACAACCTATGAAaATAGAcaaATAGTgncTGTTACTTtaaATAaanaTAAGanngg
taanaAGTTAGTCGTaTATCCATTtgaaannaAAca
SEQUENCE ID NO: 29 >APC1497.seq-ID: Pig B4 BSH-BSH1 Forward on
2014 May 28-21:57:36 automatically edited with PhredPhrap, start with
base no.: 32 Internal Params: Windowsize: 20, Goodqual: 19, Badqual:
10, Minseqlength: 50, nbadelimit: 1
AtATGGCGAgcagGTAATCATTACTCCGGCtGagtATGaATttaaaTTTaGAAAGGAAAAGGctatAAAGA
ATCATAAATCAttaaTAGgtgtTGGAATTGTCGCtGACGATTACCCAtTGTATTTTGATGCTATTAATGAG
GATGGactAGGAATGGCAGGATTGAATTTTCCTGGAAATGCATATTATAGCGAtttTTTAGAGAATGACAA
AGATAATATTACGCcntTTGAGTTTATTCCATgGATTCTGGgaCAGtgtAGCGAtgtTAATGAAGCAAGAA
ATTTAGTTGAAAGAATAAATCTCATTAATCTTAGTTTTAGCGAACAATTACCTTTAGCAGgatTACATTGg
TTAATTGcagATAGAGAAAAATCTATTGTAGTAGAAGTAACTAAATCTGGCGTACATATTTATGATAATCC
AATTGGAGTATTGACTAATAATCCGGAATTTAATTATCAGATGTataATCTGAATAAATATCGCAACTTAT
CTATCAGTACACCACAAAATACATTCTCAGATAGCGTGGATTTAAAGGTAGACGGTACTGGTTTTGGCGGT
ATTGGCTTACCAGGCGatgCATCTCCCGAATCTCGTTTTGTGAGAGCTGCTTTTAGCAAGTTAAATTCAAG
TAAAGgnaCGACCGTagAAGAAGATATTACTCAGTTTTTCCATATACTAGGGACAGTAGAACAGATAAAGG
GCGTTAATAAGaCAGAATCAGGAAAAGAAGAATATACTGTATATTCGAATTGCTATGATTTGGACAACAAA
ACGTTATATTATACAACCTATGAAAATAGACAAATAGTGTCTGTTACtttAaaTAAAGATAAGAATGGTAA
TAAGTTAGTCGTATATCCATTTGAAAGAAAACAAGTAATAAATa
SEQUENCE ID NO: 31 >APC1498.seq-ID: Pig B6 BSH-BSH1 Forward on
2014 May 28-21:57:36 automatically edited with PhredPhrap, start with
base no.: 13 Internal Params: Windowsize: 20, Goodqual: 19, Badqual:
10, Minseqlength: 50, nbadelimit: 1
TaGaTTtGgncanTTtcaTATGGCGagcaGGTAATCAttACTCCGGCTGagtATGAATTtAaATTTAGAAA
GGAAAAGGCTATAAAGAATCATAAATCATTAATAGGTGTTGGAATTGTCGCTGACGATTACCCAtTGTATT
TTGATGCTATTAATGAGGATGGACTAGGAATGGCAGGATTGAATTTTCCTGGAAATGCATATTATAGCGAT
TTTTTAgagAATGACAAAGATAATATTACGCCATTtGAGTTTATTCCATgGaTTCTGGgacAGtgtaGCGa
tgtTAATGAAGCAAGAAATTTAGTTGAAAGAATAAATCTCATTAATCTTagtTTTAGCGAACAATTACCTT
TAGCAGGGTTACATTGGTTAATTGCAGATAGAGAAAAATCTATTGTAGTAGAAGTAACTAAATCTGGCGTA
CATATTTATGATAATCCAATTGGAGTATTGACTAATAATCCGGAATTTAATTATCAGATGTATAATCTGAA
TAAATATCGCAACTTATCTATCAGTACACCACAAAATACATTCTCAGATAGCGTGGATTTAAAGGTAGACG
GTACTGGTTTTGGCGGTATTGGCTTACCAGGCGATGCATCTCCCGAATCTCGTTTTGTGAGAGCTGCTTTT
AGCAAGTTAAATTCAAGTAAAGGGACGACCGTAGAAGAAGATATTACTCAGTTtTTCCATATACTAGGGAC
AGTAGAACAGATAAAGGGCGTTAATAAGaCAGAATCAGGAAAAGAAGAATATACTGTATATTCGAATTGCT
ATGATTTGGACAACAAAACGTTATATTATACAACCTATGAAnATAGACaaaTAGTGTCTGTTACTgtAnAT
aaagATAanaATGGTAATAAGttAGTCGTATATCCATtTGAAAGanAACAAGTAATAAata
SEQUENCE ID NO: 33 >APC1499.seq-ID: Pig B9 BSH-BSH1 Forward on
2014 May 28-21:57:36 automatically edited with PhredPhrap, start with
base no.: 32 Internal Params: Windowsize: 20, Goodqual: 19, Badqual:
10, Minseqlength: 50, nbadelimit: 1
AtaTGGCGagcagGtAATCattaCTCcgGctgagtatgaGtttaaaTtTaGAAAGgaaAAGGCtataAAGA
ATCATAAATCATTaataGgtgttgGAATtgTCGctAACGAttaCCcattgTATTTtGATGCtATTAAtGaG
GATgGATTAGGAATGGCAGGATTGAATTTTCctgGAAATGCATATTATAGCGaTgCTTTagagAATGACAA
AgaTAATATTACACCGtTCGaGTTTATTCCATGGATtctGGGACAgtgnagCGAtgTTAATGAAGCAAGAA
ATTTAGttGAAAGAATAAATCTCATTAATCTTAgTTTTaGCGAACaATTACCTTTAGCAGGATTACATTgg
TTAATTGCTGATagAGAAAAATCCATTGTAGTAGAAgtaACTAAATCTGGCGTACATATTTATGATAATCC
AATTGGAGTATTGACTAATAATCCGGAATTTAATTATCAGATgtACAATCTGAATAAATATCgcAACTTAT
CTATCAGTACACCACaAAATACATTCTCAGATAGCGTGgATTTAAAGGTAgACGGTACTGgctTTTGGTGG
TATTGGCTTACCAGGCgACGTATCTCCCGAATCTCGTTTTGTGAGAGTTGCTTTTAGCAAGTTAAATTCAa
ATAAAGGAACGACCgTAGAAGAAGATATTACTCAGTTTTTCCATATACTnngGACAGtagAACAGATAAAG
GGTGTTAATAAGaCaGAATcagGAAAAGAAGAATATAcTgtATATTCgaaTTGCTAtaATTTGGACAACaa
aACGTTATatTATACAACCTATGanaATAGACaaaTAgtgTCTGTTACTtnnaatca
SEQUENCE ID NO: 35 >APC1500.seq-ID: Pig B10 BSH-BSH1 Forward on
2014 May 28-21:57:36 automatically edited with PhredPhrap, start with
base no.: 15 Internal Params: Windowsize: 20, Goodqual: 19, Badqual:
10, Minseqlength: 50, nbadelimit: 1
atTTgnacTaTTtcaTATGGCGAgcAngGTAATCAttACTCCGGCtgagtATGAATttaaATTTaGAAAGG
AAAAGGCTataAAGAATCATAAATCAtTaatAGgtgtTGGAATTGTCGCTGACgATTACCcattgTATTTT
GATGCtATTAATGAGGaTGGACTAGGAATGGCAGGATTGAATTTTCCTGGAAATGCATATTATAGCGATTT
TTTaGAGAATGACAAAGATAATATTACGCCATTTGAGTTTATTCCATgGATTCTGGgaCAGtgtagCGatg
tTAATGAAGCAAGAAATTTAGTTGAAAGAATAAATCTCATTAATCTTAGTTTTAGCGAACAATTACCTTTA
GCAGGGTTACATTgGTTAATTGCAGATAGAGAAAAATCTATTGTAGTAGAAGTAACTAAATCTGGCGTACA
TATTTATGATAATCCAATTGGAGTATTGACTAATAATCCGGAATTTAATTATCAGATGTATAATCTGAATA
AATATCGCAACTTATCTATCAGTACACCACAAAATACATTCTCAGATAGCGTGGATTTAAAGGTAGACGGT
ACTGGTTTTGGCGGTATTGGCTTACCAGGCGATGCATCTCCCGAATCTCGTTTTGTGAGAGCTGCTTTTAG
CAAGTTAAATTCAAGTAAAGGGACGACCGTAGAAGAAGATATTACTCAGTTTTTCCATATACTAGGGACAG
TAGAACAGATAAAGGGCGTTAATAAGaCAGAATCAGGAAAAGAAGAATATACTGTATATTCGAATTGCTAT
GATTTGGACAACAAAACGTTATATTATACAACCTATGAAAATAGACaaaTAGTGTcnGTTActttaaatAA
AGATAAGaATGGTAATAAGTtAGTCGTATATCCATTTGAnAGAAAACAAGTAataAATAAgnttGAATTAa
SEQUENCE ID NO: 37 >APC1501 BSH-BSH1 Forward on 2014 May 28-21:57:36
automatically edited with PhredPhrap, start with base no.: 30 Internal
Params: Windowsize: 20, Goodqual: 19, Badqual: 10, Minseqlength: 50,
nbadelimit: 1
GtaTGGCGaGcngGtaaTcattACTCCGGCtgagtntgAGTttaAATttaGAAAGGAAAAAGcTATAAAGA
ATCATAAATCAtTGATAGgtgtTgGAATtgTCGctaACGCttaCCCAttgTATTTTGATGCtattaATGAG
GATggaCtAGGAATGGCAGGATtgAATTTTCCTgGAAATGCATATTATAGCGaTgCTTTAgagAATGATAA
AGATAATATTACGCCGTTCGAGTTTATTCCATgGATTCtGGGACAgtgtaGCGatgtTAATGAAGCAAGAA
ATTTAGTTGAAAGAATAAATCTCATTAATCTTAgtTTTAGCGAACAATTACCTTTAGCAGGGTTACATTGG
TTAATTGCAGATAGAGAAAAATCCATTGTAGTAGAAGTAACTAAATCTGGCGTACATATTTATGATAATCC
AATTGGAGTATTGACTAATAATCCGGAATTTAATTATCAGATGTATAATCTGAATAAATATCGCAACTTAT
CTATCAGTACACCACAAAATACATTCTCAGATAGTGTGGATTTAAAGGTAGACGGTACTGGTTTTGGCGGT
ATTGGATTGCCAGGCGATGCATCTCCCGAATCTCGTTTTGTGAGAGCTGCTTTTAGCAAGTTAAATTCAAg
nnAAAGGGACGACCGTAGAAGAagaTATTACTCAGTTTTTCCATATACTnnnGACAGTAGaACAGATAAAG
gGcgttnntAaga
SEQUENCE ID NO: 39 >APC1502 BSH-BSH1 Forward on 2014 May 28-21:57:36
automatically edited with PhredPhrap, start with base no.: 59 Internal
Params: Windowsize: 20, Goodqual: 19, Badqual: 10, Minseqlength: 50,
nbadelimit: 1
ggtaATCantaCTCCgGCtgngtATgaatttnnatttaGAAAGgaaAagGctataAAGAATCAtAAATCAT
taataGgtgttgGaATtgtCGcTGACGAtTACCcnttGTATTTTgatgctATTAATGAGGATGgactAgga
ATGGCAGGATtgaATTTTcctgGaAATGCatATTATAGCGATTTTTTagAGAAtgACAAAGATAATATTAC
GCCATTTgaGTTTATTCCATgGATTCTGGgacAGTgtagCGAtgttaatGAAGCAAGAAATTTagTTGAAA
GAATAAATctCATTAATCTTAgttTTAGCGAACAATTACCTTTAGCAGGGtTACATTgGTTAATTGCAGAT
AGAGAAAAATCTATTGTAGTAGAAgtaACTAAATCTGGCGTACATATTTATGATAATCCAATTGGAGTATT
GACTAATAATCCGGAATTTAATTATCAGATGTATAATCTGAATAAATATCGCAACTTATCTATCAGTACAC
CACAAAATACATTCTCAGATAgCGTGGATTTAAAGGTAGACGGTACTGGtTTTGGCGGTATTGgcTTACCA
GGCGATGCAtcTCCCGAATCTCGTTTTGTGAGAGCTGCTTTTAGCAAGTTaAATTCAAGTAAAGGGACGAC
CGTAGaagaAGATATTACTCAGtttTTCCATATaCTaggGACAGTannACanaTAaagggcGTTAATAAGA
CAgAATCaggAAAagaanAATATACTGTATATTcnaAATTGCTATGATTt
16S-rRNA SEQUENCES
SEQUENCE ID NO: 4 >APC1484-DG74 on 2014 May 24-1:5:8 automatically edited
with PhredPhrap, start with base no.: 34 Internal Params: Windowsize:
20, Goodqual: 19, Badqual: 10, Minseqlength: 50, nbadelimit: 1
TCTgtcccaCCTTanACGGCTGGCTCCTTGCGGTTACCCCACCGGCTTTGGGTGTTACAAACTCTCATGGT
GTGACGGGcGGtGTGTACAAGGCCCGGGAACGTATTCACCGCGACATGCTGATTCGCGATTACnAgcGATT
CCgACTTCATGTAGGCgAgTTGCaGCCTACAATCCGAACTGAgAACGGCTTTAAgAGATTAGCTAAACCTC
GCGGTCTCGCGACTCGTTGTACCGTCCATTGtAnCAcGTGtGtagcCCAgGTCATAAGGGGcatGATGACt
TGACgTCaTCCCCAcctt
SEQUENCE ID NO: 6 >APC1485-DG74 on 2014 May 24-1:5:9 automatically
edited with PhredPhrap, start with base no.: 34 Internal Params:
Windowsize: 20, Goodqual: 19, Badqual: 10, Minseqlength: 50,
nbadelimit: 1
TCtgtcCcaCCTTanACGGCTGGCTCCTTGCGGTTACCCCACCGGCTTTGGGTGTTACAAACTCTCATGGT
GTGACGGGcGGtGTGTACAAGGCCCGGGAACGTATTCACCGCGACATGCTGATTCGCGATTACnAgCGATT
CCgACTTCATGTAGGCGAGTTGCAGCCTACAATCCGAACTGAGAACGGCTTTAAgAGATTAGCTAAACCTC
GCGGTCTCGCGACTCGTTGTACCGTCCATTGTAgCAcGTGtGtagcCCAGGTCATAAGGGGcatGATGACT
TGACGTCaTCCCCACcT
SEQUENCE ID NO: 8 >APC1486-DG74 on 2014 May 24-1:5:10 automatically
edited with PhredPhrap, start with base no.: 16 Internal Params:
Windowsize: 20, Goodqual: 19, Badqual: 10, Minseqlength: 50,
nbadelimit: 1
aCtTcncctAaTcnTCTgTCCtACCTTAGACGGCTGaCTCCtataaaGGtTaTCcCaccGGcTTTGGGTGT
TACanACTCtcnnGGTGTGACGGGCGGTGTGtacnagGCCcGggAAcgTatTCaCCGCGgCGtGcTGATCC
gcgATTACnAgcGAttccngcTtCGTGtangcnAgTTgcanCCTAca
SEQUENCE ID NO: 10 >APC1487 rRNA-DG74 on 2014 May 28-21:54:26
automatically edited with PhredPhrap, start with base no.: 30 Internal
Params: Windowsize: 20, Goodqual: 19, Badqual: 10, Minseqlength: 50,
nbadelimit: 1
CcaaTCnTCTGTCCcaCCTTAGACGGCTGGCTCCTTGCGGTTACCCCACCGGCTTTGGGTGTTACAAACTC
TCATGGTGTGACGGGCGGTGTGTACAAGGCCCGGGAACGTATTCACCGCGACATGCTGATTCGCGATTACT
AGCGATTCCGACTTCATGTAGGCGAGTTGCAGCCTACAATCCGAACTGAGAACGGCTTTAAGAGATTAGCT
AAACCTCGCGGTCTCGCGACTCGTTGTACCGTCCATTGTAgCACGTGTGTAgCCCAGGTCATAAGGGGCAT
GATGACTTGACGTCATCCCCACCTTcctCCAGTTa
SEQUENCE ID NO: 12 >APC1488-DG74 on 2014 May 24-1:5:5 automatically
edited with PhredPhrap, start with base no.: 46 Internal Params:
Windowsize: 20, Goodqual: 19, Badqual: 10, Minseqlength: 50,
nbadelimit: 1
acGGcTGGcTCCTTGCGGttaCcccaccGGctTTGGGTGTTACAAACTCTCAtGGtGTGacggggggtGtG
tacaAgGcCCGGGAAcgtATTCaccGCGACATGCTGATTCgCGATTAcnancGattccnACtTCaTGTAgG
CgAgttgcagCCtACaATCCgAACTGAgAAcGGcTTtAAaAgATtAgCTAAACCTCGCGGtCTCGCgACtC
gTTGTACCGTccatTGtAacangtgtgtancCcAgGtcAtAAgGggcaTGATGACtTGacntcntcccCa
SEQUENCE ID NO: 14 >APC1489-DG74 on 2014 May 24-1:5:6 automatically
edited with PhredPhrap, start with base no.: 43 Internal Params:
Windowsize: 20, Goodqual: 19, Badqual: 10, Minseqlength: 50,
nbadelimit: 1
caCcttnnacGGcTGGCTCCTTGCGGTTACCCCacCGGCTTTGGGTGTTACAAACTCTCATGGTGTGACgG
GcGGtGTGtACaAGGCCCGGGAACGTATTCaCCGCGACATGCTGATTCgCGATTACnAgcGATTCCgACTT
CATGTAGGCgAgTTGcagCCtACAATCCGAACTGAgAACGGCTTTAAgAGATTAgCTAAACCTCGcGGTCT
CGCgACTCGTTGTACCGTccatTGtAnCAcgTGtGtagcCCAGGtCATAAGGggcatGATGACtTGACgtC
aTCCCcanct
SEQUENCE ID NO: 16 >APC1490-DG74 on 2014 May 24-1:5:6 automatically
edited with PhredPhrap, start with base no.: 24 Internal Params:
Windowsize: 20, Goodqual: 19, Badqual: 10, Minseqlength: 50,
nbadelimit: 1
CCCnaaTcnTCTgTCCCaCCTTAGACGGCTGGCTCCTTGCGGTTACCCCACCGGCTTTGGGTGTTACAAAC
TCTCATGGTGTGACGGGCGGTGTGTACAAGGCCCGGGAACGTATTCACCGCGACATGCTGATTCGCGATTA
CtAGCGATTCCGACTTCATGTAGGCGAGTTGCAGCCTACAATCCGAACTGAGAACGGCTTTAAgAGATTAg
CTAAACCTCGCGGTCTCGCGACTCGTTGTACCGTCCATTGTAgCACGTGTGTAgCCCAGGTCATAAGGGGC
ATGATGACtTGACgTCATCCCCACCT
SEQUENCE ID NO: 18 >APC1491-DG74 on 2014 May 24-1:5:7 automatically
edited with PhredPhrap, start with base no.: 30 Internal Params:
Windowsize: 20, Goodqual: 19, Badqual: 10, Minseqlength: 50,
nbadelimit: 1
TcnTCTgtCCcaCCTTAgacGGCTGGCTCCTTGCGGTTACCCCACCGGCTTTGGGTGTTACAAACTCTCAT
GGTGTGACGGGCGGTGTGTACAAGGCCCGGGAACGTATTCACCGCGACATGCTGATTCGCGATTACtAgCG
ATTCCgACTTCATGTAGGCGAGTTGCAGCCTACAATCCGAACTGAGAACGGCTTTAAgAGATTAGCTAAAC
CTCGCGGTCTCGCGACTCGTTGTACCGTCCATTGTAgCACGTGTGTAgCCCAGGTCATAAGGGGCATGATG
ACTTGACgTCATCCCCACcttnna
SEQUENCE ID NO: 20 >APC1492 rRNA-DG74 on 2014 May 28-21:54:27
automatically edited with PhredPhrap, start with base no.: 30 Internal
Params: Windowsize: 20, Goodqual: 19, Badqual: 10, Minseqlength: 50,
nbadelimit: 1
cnTCTGTCCcaCCTTAGACGGCTGGCTCCTTGCGGTTACCCCACCGGCTTTGGGTGTTACAAACTCTCATG
GTGTGACGGGCGGTGTGTACAAGGCCCGGGAACGTATTCACCGCGACATGCTGATTCGCGATTACTAGCGA
TTCCGACTTCATGTAGGCGAGTTGCAGCCTACAATCCGAACTGAGAACGGCTTTAAGAGATTAGCTAAACC
TCGCGGTCTCGCGACTCGTTGTACCGTCCATTGTAGCACGTGTGTAGCCCAGGTCATAAGGGGCATGATGA
CTTGACGTCATCCC
SEQUENCE ID NO: 22 >APC1493 rRNA-DG74 on 2014 May 28-21:54:28
automatically edited with PhredPhrap, start with base no.: 25 Internal
Params: Windowsize: 20, Goodqual: 19, Badqual: 10, Minseqlength: 50,
nbadelimit: 1
CCCcnaTCnTCTGTCCcaCCTTAGACGGCTGGCTCCTTGCGGTTACCCCACCGGCTTTGGGTGTTACAAAC
TCTCATGGTGTGACGGGCGGTGTGTACAAGGCCCGGGAACGTATTCACCGCGACATGCTGATTCGCGATTA
CTAGCGATTCCGACTTCATGTAGGCGAGTTGCAGCCTACAATCCGAACTGAGAACGGCTTTAAGAGATTAG
CTAAACCTCGCGGTCTCGCGACTCGTTGTACCGTCCATTGTAGCACGTGTGTAGCCCAGGTCATAAGGGGC
ATGATGACTTGACGTCATCCCC
SEQUENCE ID NO: 24 >APC1494 rRNA-DG74 on 2014 May 30-3:53:23
automatically edited with PhredPhrap, start with base no.: 15 Internal
Params: Windowsize: 20, Goodqual: 19, Badqual: 10, Minseqlength: 50,
nbadelimit: 1
cgAcTTcnCCCcaATcaTCTGTCCcaCCTTAGACGGCTGGCTCCTTGCGGTTACCCCACCGGCTTTGGGTG
TTACAAACTCTCATGGTGTGACGGGCGGTGTGTACAAGGCCCGGGAACGTATTCACCGCGACATGCTGATT
CGCGATTACTAGCGATTCCGACTTCATGTAGGCGAGTTGCAGCCTACAATCCGAACTGAGAACGGCTTTAA
GAGATTAGCTAAACCTCGCGGTCTCGCGACTCGTTGTACCGTCCATTGTAGCACGTGTGTAGCCCAGGTCA
TAAGGGGCATGATGACTTGACGTCATCCCCACCTTc
SEQUENCE ID NO: 26 >APC1495 rRNA-DG74 on 2014 May 30-3:53:24
automatically edited with PhredPhrap, start with base no.: 30 Internal
Params: Windowsize: 20, Goodqual: 19, Badqual: 10, Minseqlength: 50,
nbadelimit: 1
tcnTCTgTCCcacCTTAgACGGCTGgcTCCtTGcggntaCccCaCcGgcttTgggtGttaca
SEQUENCE ID NO: 28 >APC1496 rRNA-DG74 on 2014 May 30-3:53:24
automatically edited with PhredPhrap, start with base no.: 30 Internal
Params: Windowsize: 20, Goodqual: 19, Badqual: 10, Minseqlength: 50,
nbadelimit: 1
tcnTCTGTCCcaCCTTAGACGGCTGGCTCCTTGCGGTTACCCCACCGGCTTTGGGTGTTACAAACTCTCAT
GGtGTGACGGGCGGTGTGTACAAGGCCCGGGAACGTATTCACCGCGACATGCTGATTCGCGATTACTAGCG
ATTCCgACTTCATGTAGGCGAGTTGCAGCCTACAATCCGAACTGAGAACGGCTTTAAgAGATTAGCTAAAC
CTCGCGGTCTCGCGACTCGTTGTACCGTCCATTGTAgCACGTGTGTAgCCCAGGTCATAAGGGGCATGATG
ACTTGACGTCATCCCCACCTtccnccngTTAta
SEQUENCE ID NO: 30 >APC1497 rRNA-DG74 on 2014 May 30-3:53:25
automatically edited with PhredPhrap, start with base no.: 24 Internal
Params: Windowsize: 20, Goodqual: 19, Badqual: 10, Minseqlength: 50,
nbadelimit: 1
CCCcanTcnTCTgTCCCACCTTAgACGGCTGGCTCCTTGCGGTTACCCCACCGGCTTTGGGTGTTACAAAC
TCTCATGGtGTGACGGGCGGTGTGTACAAGGCCCGGGAACGTATTCACCGCGACATGCTGATTCgCGATTA
CTAGCGATTCCgACTTCaTGTAGGCGAGTTGCAgCCTACAATCCGAACTGAGAACGGCTTTAAgAGATTAg
CTAAACCTCGCGGTCTCGCGACTCGTTGTACCGTCCATTGTAgCACGTGTGTAgCCCAgGTCATAAgGGGC
ATGATGACTTGACgTCaTCCCCaCCTt
SEQUENCE ID NO: 32 >APC1498 rRNA-DG74 on 2014 May 30-3:53:25
automatically edited with PhredPhrap, start with base no.: 16 Internal
Params: Windowsize: 20, Goodqual: 19, Badqual: 10, Minseqlength: 50,
nbadelimit: 1
tacgAcTTcnCCCcaaTcnTCTGTCCcaCCTTAGACGGCTGGCTCCTTGCGGTTACCCCACCGGCTTTGGG
TGTTACAAACTCTCATGGTGTGACGGGCGGTGTGTACAAGGCCCGGGAACGTATTCACCGCGACATGCTGA
TTCGCGATTACTAGCGATTCCGACTTCATGTAGGCGAGTTGCAGCCTACAATCCGAACTGAGAACGGCTTT
AAGAGATTAGCTAAACCTCGCGGTCTCGCGACTCGTTGTACCGTCCATTGTAGCACGTGTGTAGCCCAGGT
CATAAGGGGCATGATGACTTGACGTCATCCCCACCTt
SEQUENCE ID NO: 34 >APC1499 rRNA-DG74 on 2014 May 30-3:53:21
automatically edited with PhredPhrap, start with base no.: 18 Internal
Params: Windowsize: 20, Goodqual: 19, Badqual: 10, Minseqlength: 50,
nbadelimit: 1
gACTTcnCCCcaaTcaTCtGTCCcaCCTTAGACGGCTGGCTCCTTGCGGTTACCCCACCGGCTTTGGGTGT
TACAAACTCTCATGGTGTGACGGGCGGTGTGTACAAGGCCCGGGAACGTATTCACCGCGACATGCTGATTC
GCGATTACTAGCGATTCCGACTTCATGTAGGCGAGTTGCAGCCTACAATCCGAACTGAGAACGGCTTTAAG
AGATTAGCTAAACCTCGCGGTCTCGCGACTCGTTGTACCGTCCATTGTAGCACGTGTGTAGCCCAGGTCAT
AAGGGGCATGATGACTTGACGTCATCCCCACCTtncnccAGTTA
SEQUENCE ID NO: 36 >APC1500 rRNA-DG74 on 2014 May 30-3:53:22
automatically edited with PhredPhrap, start with base no.: 30 Internal
Params: Windowsize: 20, Goodqual: 19, Badqual: 10, Minseqlength: 50,
nbadelimit: 1
TCtgtCcnaccTTanACGGCtGgcTCCTTGcngntacc
HUMAN rRNA
SEQUENCE ID NO: 38 >APC1501 rRNA-DG74 on 2014 May 24-1:5:8 automatically
edited with PhredPhrap, start with base no.: 20 Internal Params:
Windowsize: 20, Goodqual: 19, Badqual: 10, Minseqlength: 50,
nbadelimit: 1
AcTTcnCCCnaaTcaTTTgtCCcaCCTTCGACGGCTAGCTCCaAATGGTTACTCCACCGGCTTCGGGTGTT
ACAAACTCTCGTGGTGTGACGGGCGGTGTGTACAAGACCCGGGAACGTATTCACCGTAGCATGCTGATCTA
CGATTACTAGCGATTCCAGCTTCATATAGTCGAGTTGCAGACTACAATCCGAACTGAGAACAACTTTATGG
GATTTGCTTGACCTCGCGGTTTCgCTGCCCTTTGTATTGTCCATTGTAGCACGTGTGTAGCCCAAATCATA
AGGGGCATGATGATTTGACGTCATCCCCA
SEQUENCE ID NO: 40 >APC1502 rRNA-DG74 on 2014 May 24-1:5:9
automatically edited with PhredPhrap, start with base no.: 33 Internal
Params: Windowsize: 20, Goodqual: 19, Badqual: 10, Minseqlength: 50,
nbadelimit: 1
TcaTCTATCCcnCCTTAGGCGGCTGGCTCCaAAAGGtTACCTCACCGACTTCGGGTGTTACAAACTCTCGT
GGTGTGACGGGCGGTGTGTACAAGGCCCGGGAACGTATTCACCGCGGCGTGCTGATCCGCGATTACTAGCG
ATTCCGACTTCATGTAGGCGAGTTGCAGCCTACAATCCGAACTGAGATTGGCTTTAAGAGATTAGCTTGCC
GTCACCGACTCGCAACTCGTTGTACCAACCATTGTAGCACGTGTGTAGCCCAGGTCATAAGGGGCATGATG
ATTTGACGTCATCCCCACCTTCc

1. A non-therapeutic method of reducing weight gain, serum cholesterol levels, or liver triglyceride levels, in a non-obese mammal, or a method of treating or preventing obesity, hypercholesterolemia or a disease or condition associated with dysregulated circadian rhythm, comprising the step of administering to the gut of a mammal an active agent comprising a bacteria that expresses BSH1 enzyme of SEQUENCE ID NO: 1, or a functional variant thereof having at least 90% sequence identity with SEQUENCE ID NO: 1.

2. A method according to claim 1 in which the active agent comprises a bacteria that expresses BSH1 enzyme of SEQUENCE ID NO: 1, or a functional variant thereof having at least 96% sequence identity with SEQUENCE ID NO: 1.

3. A method as claimed in claim 1 in which the active agent is administered to the gut of the mammal as part of a formulation suitable for oral delivery.

4. A method as claimed in claim 3 in which the formulation is a food product.

5. A method as claimed in claim 1 in which the bacteria is a wild-type bacteria.

6. A method as claimed in claim 1 in which the bacteria is genetically modified to express BSH1 enzyme of SEQUENCE ID NO: 1, or the functional variant thereof.

7. A method as claimed in claim 1 in which the bacteria is a probiotic bacteria

8. A method as claimed in claim 1, in which the bacteria is capable of elevated expression of BHS1 enzyme or the functional variant thereof.

9-28. (canceled)

29. A method as claimed in claim 1 in which the disease or condition associated with dysregulated circadian rhythm is sleep apnoea.


SEQUENCE LISTING (46 sequences)

SEQ ID NO: 1 - protein, 324 aa, Lactobacillus salivarius
MCTAITLNGNSNYFGRNLDLDFSYGEQVIITPAEYEFKFRKEKAIKNHKSLIGVGIVANAYPLYFDAINEDGLGMAGLNFPGNAYYSDALENDKDNITPFEFIPWILRQCSDVNEARNLVERINLINLSFSEQLPLAGLHWLIADREKSIVVEVTKSGVHIYDNPIGVLTNNPEFNYQMYNLNKYRNLSISTPQNTFSDSVDLKVDGTGFGGIGLPGDVSPESRFVRAAFSKLNSSKGTTVEEDITQFFHILGTVEQIKGVNKTESGKEEYTVYSNCYDLDNKTLYYTTYENRQIVAVTLNEDKNGNGLIAYPFERKQVINKLN

SEQ ID NO: 2 - DNA, 975 bp, Lactobacillus salivarius
atgtgtacagcaattactttaaatggtaatagtaattattttggaagaaatttagatttggatttttcatatggcgagcaggtaatcattactccggctgagtatgagtttaaatttagaaaggaaaaagctataaagaatcataaatcattgataggtgttggaattgtcgctaacgcttacccattgtattttgatgctattaatgaggatggactaggaatggcaggattgaattttcctggaaatgcatattatagcgatgctttagagaatgataaagataatattacgccgttcgagtttattccatggattctgagacagtgtagcgatgttaatgaagcaagaaatttagttgaaagaataaatctcattaatcttagttttagcgaacaattacctttagcagggttacattggttaattgcagatagagaaaaatccattgtagtagaagtaactaaatctggcgtacatatttatgataatccaattggagtattgactaataatccggaatttaattatcagatgtacaatctgaataaatatcgcaacttatctatcagtacaccacaaaatacattctcagatagcgtggatttaaaagtagacggtaccggttttggtggtattggcttaccaggcgatgtatctcccgaatctcgttttgtgagagctgcttttagcaagttaaattcaagtaaagggacgaccgtagaagaagatattactcagttttttcatatactagggacagtagaacagataaagggcgttaataagacagaatcaggaaaagaagaatatactgtatattcgaattgttatgatttggacaacaagacgttatattatacaacctatgaaaatagacaaatagtagctgttactttaaatgaagataagaatggtaatgggttaattgcatatccatttgaaagaaaacaagtaataaataagttgaattaa

SEQ ID NO: 3 - DNA, 934 bp, Lactobacillus salivarius
  misc_feature (8)..(8): n is a, c, g, or t
ttggagancntagatttggactttncatatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagacagaatcaggaaaagaagaatatactgtatattcgaattgctatgatttggacaacanaacgttatattatacaacctatgaanatagacaaatagtgtctgttactttanataaagataagaatggtaataagttagtcgtatatccatttgaaaganaacaagtaataaatagnttgaattaat

SEQ ID NO: 4 - DNA, 302 bp, Lactobacillus salivarius
  misc_feature (15)..(15): n is a, c, g, or t
tctgtcccaccttanacggctggctccttgcggttaccccaccggctttgggtgttacaaactctcatggtgtgacgggcggtgtgtacaaggcccgggaacgtattcaccgcgacatgctgattcgcgattacnagcgattccgacttcatgtaggcgagttgcagcctacaatccgaactgagaacggctttaagagattagctaaacctcgcggtctcgcgactcgttgtaccgtccattgtancacgtgtgtagcccaggtcataaggggcatgatgacttgacgtcatccccacctt

SEQ ID NO: 5 - DNA, 917 bp, Lactobacillus salivarius
  misc_feature (2)..(2): n is a, c, g, or t
cntagatttggatttttcatatggcgagcaggtaatcattactccggctgagtatgagtttaaatttagaaaggaaaaggctataaagaatcataaatcattaataggtgttggaattgtcgctaacgattacccattgtattttgatgctattaatgaggatggattaggaatggcaggattgaattttcctggaaatgcatattatagcgatgctttagagaatgacaaagataatattacaccgttcgagtttattccatggattctgggacagtgtagcgatgttaatgaagcaagaaatttagttgaaagaataaatctcattaatcttagttttagcgaacaattacctttagcaggattacattggttaattgctgatagagaaaaatccattgtagtagaagtaactaaatctggcgtacatatttatgataatccaattggagtattgactaataatccggaatttaattatcagatgtacaatctgaataaatatcgcaacttatctatcagtacaccacaaaatacattctcagatagcgtggatttaaaggtagacggtactggttttggtggtattggcttaccaggcgacgtatctcccgaatctcgttttgtgagagttgcttttagcaagttaaattcaaataaaggaacgaccgtagaagaagatattactcagtttttccatatactagggacagtagaacagataaagggtgttaataagacagaatcaggaaaagaagaatatactgtatattcgaattgctataattnggacaacnnaacgttatattatacaacctatgaaaatagaccaatagtgtctgttactttanataaagataagaatggtaataagttagtcgtatatccatttgnnagaaaacaagtaanaaataggt

SEQ ID NO: 6 - DNA, 301 bp, Lactobacillus salivarius
  misc_feature (15)..(15): n is a, c, g, or t
tctgtcccaccttanacggctggctccttgcggttaccccaccggctttgggtgttacaaactctcatggtgtgacgggcggtgtgtacaaggcccgggaacgtattcaccgcgacatgctgattcgcgattacnagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccacct

SEQ ID NO: 7 - DNA, 927 bp, Lactobacillus johnsonii
  misc_feature (8)..(8): n is a, c, g, or t
tttggagnacntagatttgganttttcatatggcgagcaggtaatcattactccggctgagtatgagtttaaatttagaaaggaaaaggctataaagaatcataaatcattaataggtgttggaattgtcgcnnacgattacccattgtattttgatgctattaatgaggatggattaggaatggcaggattgaattttcctggaaatgcatattatagcgatnntttagagaatgacaaagataatattacaccgttcgagtttattccatggattctgggacagtgtagcgatgttaatgaagcaagaaatttagttgaaagaataaatctcattaatcttagttttagcgaacaattacctttagcaggattacattggttaattgctgatagagaaaaatcnattgtagtagaagtaactaaatctggcgtacatatttatgataatccaattggagtattgactaataatccggaatttaattatcagatgtacaatctgaataaatatcgcaacttatctatcagtacaccacaaaatacattctcagatagcgtggatttaaaggtagacggtactggttttggtggtattggcttaccaggcgacgtatctcccgaatctcgttttgtgagagntgcttttagcaagttaaattcaaataaaggaacgaccgtagaagaagatattactcagtttttccatatactagggacagtagaacagataaagggngttaataagacagaatcaggaaaagaagaatatactgtatattcgaattgctatgatttggacaacanaacgttatattatacaacctatgaaaatagacaaatagtgtctgttactttaaataaagataagaatggtaataagttagtcgtatatccatttgaaagaaaacaagtaataaatagnnt

SEQ ID NO: 8 - DNA, 189 bp, Lactobacillus johnsonii
  misc_feature (6)..(6): n is a, c, g, or t
acttcncctaatcntctgtcctaccttagacggctgactcctataaaggttatcccaccggctttgggtgttacanactctcnnggtgtgacgggcggtgtgtacnaggcccgggaacgtattcaccgcggcgtgctgatccgcgattacnagcgattccngcttcgtgtangcnagttgcancctaca

SEQ ID NO: 9 - DNA, 867 bp, Lactobacillus salivarius
  misc_feature (4)..(4): n is a, c, g, or t
ttcntatggcgagcaggtaatcattactccggctgagtatgagtttaaatttagaaaggaaaaggctataaagaatcataaatcattaataggtgttggaattgtcgctaacgattacccattgtattttgatgctattaatgaggatggattaggaatggcaggattgaattttcctggaaatgcatattatagcgatgctttagagaatgacaaagataatattacaccgttcgagtttattccatggattctgggacagtgtagcgatgttaatgaagcaagaaatttagttgaaagaataaatctcattaatcttagttttagcgaacaattacctttagcaggattacattggttaattgctgatagagaaaaatccattgtagtagaagnaactaaatctggcgtacatatttatgataatccaattggagtattgactaataatccggaatttaattatcagatgtacaatctgaataaatatcgcaacttatctatcagtacaccacaaaatacattctcagatagcgtggatttaaaggtagacggtactggttttggtggtattggcttaccaggcgacgtatctcccgaatctcgttttgtgagagttgcttttagcaagttaaattcaaataaaggaacgaccgtagaagaagatattactcagtttttccatatactagggacagtanaacagataaagggtgttaataagacagaatcaggaaaagaagaatatactgnatattcgaattgctataatttggacaacaaaacgttatattatacaacctatgaaaatnnacaaatagtgtctgttactttanataaagataagaatggtaataagttagtcgt

SEQ ID NO: 10 - DNA, 319 bp, Lactobacillus salivarius
  misc_feature (7)..(7): n is a, c, g, or t
ccaatcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccaccttcctccagtta

SEQ ID NO: 11 - DNA, 883 bp, Lactobacillus salivarius
  misc_feature (805)..(805): n is a, c, g, or t
gctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagacagaatcaggaaaagaagaatatactgtatattcgaattgctatgatttggacaacaaaacgttatattatacaacctatgaaaatagacaaatagtgtctgttactttanataaagataagaatggtaataagttagtcgtatatccatttgaaaganaacaagtaataaataagtttgaattaaaaa

SEQ ID NO: 12 - DNA, 283 bp, Lactobacillus salivarius
  misc_feature (120)..(120): n is a, c, g, or t
acggctggctccttgcggttaccccaccggctttgggtgttacaaactctcatggtgtgacggggggtgtgtacaaggcccgggaacgtattcaccgcgacatgctgattcgcgattacnancgattccnacttcatgtaggcgagttgcagcctacaatccgaactgagaacggctttaaaagattagctaaacctcgcggtctcgcgactcgttgtaccgtccattgtaacangtgtgtancccaggtcataaggggcatgatgacttgacntcntcccca

SEQ ID NO: 13 - DNA, 927 bp, Lactobacillus salivarius
  misc_feature (7)..(7): n is a, c, g, or t
ttggagnacttagatttggactttnnatatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagacagaatcaggaaaagaagaatatactgtatattcgaattgctatgatttggacaacaaaacgttatattatacaacctatgaaaatagacaaatagtgtctgttactttanataaagataagaatggtaataagttagtcgtatatccatttgaaagaaaacaagtaataaatangntt

SEQ ID NO: 14 - DNA, 294 bp, Lactobacillus salivarius
  misc_feature (7)..(8): n is a, c, g, or t
caccttnnacggctggctccttgcggttaccccaccggctttgggtgttacaaactctcatggtgtgacgggcggtgtgtacaaggcccgggaacgtattcaccgcgacatgctgattcgcgattacnagcgattccgacttcatgtaggcgagttgcagcctacaatccgaactgagaacggctttaagagattagctaaacctcgcggtctcgcgactcgttgtaccgtccattgtancacgtgtgtagcccaggtcataaggggcatgatgacttgacgtcatccccanct

SEQ ID NO: 15 - DNA, 294 bp, Lactobacillus salivarius; identical to SEQ ID NO: 14
  misc_feature (7)..(8): n is a, c, g, or t

SEQ ID NO: 16 - DNA, 310 bp, Lactobacillus salivarius
  misc_feature (4)..(4): n is a, c, g, or t
cccnaatcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccacct

SEQ ID NO: 17 - DNA, 915 bp, Lactobacillus salivarius
  misc_feature (2)..(2): n is a, c, g, or t
cntagatttggacttttcatatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagncagaatcaggaaaagaagaatatactgtatattcgaattgctatgatttggacaacaaaacgttatattatacaacctatgaaaatagacaaatagtgtctgttactttnnataaagataagaatggtaataagttagtcgtatatccatttgaaagaaaacaagtaatnaatag

SEQ ID NO: 18 - DNA, 308 bp, Lactobacillus salivarius
  misc_feature (3)..(3): n is a, c, g, or t
tcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccaccttnna

SEQ ID NO: 19 - DNA, 907 bp, Lactobacillus salivarius
  misc_feature (712)..(712): n is a, c, g, or t
gatttggacttttcatatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtanaacagataaagggcgttaataagacagaatcaggaaaagaagaatatactgtatattcgaattgctatgatttggacaacaaaacgttatattatacaacctatgaaaatagacaaatagtgtctgttactttaaatnaagataagaatggtaataagntagtcgtatatccatttgaaagaaaacaagtaataa

SEQ ID NO: 20 - DNA, 298 bp, Lactobacillus salivarius
  misc_feature (2)..(2): n is a, c, g, or t
cntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccc

SEQ ID NO: 21 - DNA, 926 bp, Lactobacillus salivarius
  misc_feature (7)..(7): n is a, c, g, or t
ttggagnacntagatttggactttncanatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagngtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagnttttccatatactagggacagtagaacagataaagggcgttaataaganngaatcaggaaaagaagaatatactgtatattcgaattgctatgatttggacaacaaaacgttatattatacaacctatgaaaatagacnnatagtgtctgttactnnnnataaagataagaatggtaataagttagtcgtatatccatttgaaagaaaacaagtaataaataggtt

SEQ ID NO: 22 - DNA, 306 bp, Lactobacillus salivarius
  misc_feature (5)..(5): n is a, c, g, or t
ccccnatcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatcccc

SEQ ID NO: 23 - DNA, 927 bp, Lactobacillus salivarius
  misc_feature (11)..(11): n is a, c, g, or t
gatggagaacntagatttggactttncanatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagngnagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtanaagnagatattactcagnttttccatatactagggacagtnnaacagataaagggcgttaataaganngaatcnggaaaagaagaatatactgtatattcgaattgctatgatttggacaacaaaacgttatattatacaacctatgaaaatagacaaatagtgtctgttactnnnnntnangataagaatgntaataagttagtcgtatatccatttgaaagaaaacaagtaataaatagnnt

SEQ ID NO: 24 - DNA, 320 bp, Lactobacillus salivarius
  misc_feature (8)..(8): n is a, c, g, or t
cgacttcnccccaatcatctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccaccttc

SEQ ID NO: 25 - DNA, 937 bp, Lactobacillus salivarius
  misc_feature (9)..(9): n is a, c, g, or t
tttggagancntagatttggactttnnatatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagacagaatcaggaaaagaagaatatactgtatattcgaattgctatgatttggacaacaaaacgttatattatacaacctatgaaaatagacaaatagtgtctgttactttanataaagataagaatggtaataagttagtcgtatatccatttganagaaaacaagtaataaataagtttgaattaaaa

SEQ ID NO: 26 - DNA, 62 bp, Lactobacillus salivarius
  misc_feature (3)..(3): n is a, c, g, or t
tcntctgtcccaccttagacggctggctccttgcggntaccccaccggctttgggtgttaca

SEQ ID NO: 27 - DNA, 888 bp, Lactobacillus salivarius
  misc_feature (3)..(3): n is a, c, g, or t
ttngtatggcgagcnggtaatcattactccggctgagtatgagtttaaatttagaaaggaaaaggctataaagaatcataaatcattaataggtgttggaattgtcgctaacgattacccattgtattttgatgctattaatgaggatggattaggaatggcaggattgaattttcctggaaatgcatattatagcgatgctttagagaatgacaaagataatattacaccgttcgagtttattccatggattctgggacagngnagcgatgttaatgaagcaagaaatttanttgaaagaataaatctcattaatcttagttttagcgaacaattacctttagcaggattacattggttaattgctgatagagaaaaatccattgtagtagaagtaactaaatctggcgtacatatttatgataatccaattggagtattgactaataatccggaatttaattatcagatgtacaatctgaataaatatcgcaacttatctatcagtacaccacaaaatacattctcagatagcgtggatttaaaggtagacggtactggttttggtggtattggcttaccaggcgacgtatctcccgaatctcgttttgtgagagttgcttttagcaagntaaattcaaataaaggaacgaccgtagaagaagatattactcagtttttccatatactagggacagtagaacagataaagggtgttaataagacagaatcaggaaaagaagaatatactgtatattcgaattgctataatttggacaacaaaacgttatattatacaacctatgaaaatagacaaatagtgnctgttactttaaataaanataagannggtaanaagttagtcgtatatccatttgaaannaaaca

SEQ ID NO: 28 - DNA, 317 bp, Lactobacillus salivarius
  misc_feature (3)..(3): n is a, c, g, or t
tcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccaccttccnccngttata

SEQ ID NO: 29 - DNA, 896 bp, Lactobacillus salivarius
  misc_feature (229)..(229): n is a, c, g, or t
atatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccntttgagtttattccatggattctgggacagtgtagcgatgttaatgaagcaagaaatttagttgaaagaataaatctcattaatcttagttttagcgaacaattacctttagcaggattacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaaggnacgaccgtagaagaagatattactcagtttttccatatactagggacagtagaacagataaagggcgttaataagacagaatcaggaaaagaagaatatactgtatattcgaattgctatgatttggacaacaaaacgttatattatacaacctatgaaaatagacaaatagtgtctgttactttaaataaagataagaatggtaataagttagtcgtatatccatttgaaagaaaacaagtaataaata

SEQ ID NO: 30 - DNA, 311 bp, Lactobacillus salivarius
  misc_feature (6)..(6): n is a, c, g, or t
ccccantcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccacctt

SEQ ID NO: 31 - DNA, 913 bp, Lactobacillus salivarius
  misc_feature (10)..(10): n is a, c, g, or t
tagatttggncantttcatatggcgagcaggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagacagaatcaggaaaagaagaatatactgtatattcgaattgctatgatttggacaacaaaacgttatattatacaacctatgaanatagacaaatagtgtctgttactgtanataaagataanaatggtaataagttagtcgtatatccatttgaaaganaacaagtaataaata

SEQ ID NO: 32 - DNA, 321 bp, Lactobacillus salivarius
  misc_feature (10)..(10): n is a, c, g, or t
tacgacttcnccccaatcntctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccacctt

SEQ ID NO: 33 - DNA, 838 bp, Lactobacillus salivarius
  misc_feature (262)..(262): n is a, c, g, or t
atatggcgagcaggtaatcattactccggctgagtatgagtttaaatttagaaaggaaaaggctataaagaatcataaatcattaataggtgttggaattgtcgctaacgattacccattgtattttgatgctattaatgaggatggattaggaatggcaggattgaattttcctggaaatgcatattatagcgatgctttagagaatgacaaagataatattacaccgttcgagtttattccatggattctgggacagtgnagcgatgttaatgaagcaagaaatttagttgaaagaataaatctcattaatcttagttttagcgaacaattacctttagcaggattacattggttaattgctgatagagaaaaatccattgtagtagaagtaactaaatctggcgtacatatttatgataatccaattggagtattgactaataatccggaatttaattatcagatgtacaatctgaataaatatcgcaacttatctatcagtacaccacaaaatacattctcagatagcgtggatttaaaggtagacggtactggcttttggtggtattggcttaccaggcgacgtatctcccgaatctcgttttgtgagagttgcttttagcaagttaaattcaaataaaggaacgaccgtagaagaagatattactcagtttttccatatactnnggacagtagaacagataaagggtgttaataagacagaatcaggaaaagaagaatatactgtatattcgaattgctataatttggacaacaaaacgttatattatacaacctatganaatagacaaatagtgtctgttacttnnaatca

SEQ ID NO: 34 - DNA, 328 bp, Lactobacillus salivarius
  misc_feature (7)..(7): n is a, c, g, or t
gacttcnccccaatcatctgtcccaccttagacggctggctccttgcggttaccccaccggctttgggtgttacaaactctcatggtgtgacgggcggtgtgtacaaggcccgggaacgtattcaccgcgacatgctgattcgcgattactagcgattccgacttcatgtaggcgagttgcagcctacaatccgaactgagaacggctttaagagattagctaaacctcgcggtctcgcgactcgttgtaccgtccattgtagcacgtgtgtagcccaggtcataaggggcatgatgacttgacgtcatccccaccttncnccagtta

SEQ ID NO: 35 - DNA, 923 bp, Lactobacillus salivarius
  misc_feature (6)..(6): n is a, c, g, or t
atttgnactatttcatatggcgagcanggtaatcattactccggctgagtatgaatttaaatttagaaaggaaaaggctataaagaatcataaatcattaataggtgttggaattgtcgctgacgattacccattgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtagaacagataaagggcgttaataagacagaatcaggaaaagaagaatatactgtatattcgaattgctatgatttggacaacaaaacgttatattatacaacctatgaaaatagacaaatagtgtcngttactttaaataaagataagaatggtaataagttagtcgtatatccatttganagaaaacaagtaataaataagnttgaattaa

SEQ ID NO: 36 - DNA, 38 bp, Lactobacillus salivarius
  misc_feature (8)..(8): n is a, c, g, or t
tctgtccnaccttanacggctggctccttgcngntacc

SEQ ID NO: 37 - DNA, 723 bp, Staphylococcus epidermidis
  misc_feature (12)..(12): n is a, c, g, or t
gtatggcgagcnggtaatcattactccggctgagtntgagtttaaatttagaaaggaaaaagctataaagaatcataaatcattgataggtgttggaattgtcgctaacgcttacccattgtattttgatgctattaatgaggatggactaggaatggcaggattgaattttcctggaaatgcatattatagcgatgctttagagaatgataaagataatattacgccgttcgagtttattccatggattctgggacagtgtagcgatgttaatgaagcaagaaatttagttgaaagaataaatctcattaatcttagttttagcgaacaattacctttagcagggttacattggttaattgcagatagagaaaaatccattgtagtagaagtaactaaatctggcgtacatatttatgataatccaattggagtattgactaataatccggaatttaattatcagatgtataatctgaataaatatcgcaacttatctatcagtacaccacaaaatacattctcagatagtgtggatttaaaggtagacggtactggttttggcggtattggattgccaggcgatgcatctcccgaatctcgttttgtgagagctgcttttagcaagttaaattcaagnnaaagggacgaccgtagaagaagatattactcagtttttccatatactnnngacagtagaacagataaagggcgttnntaaga

SEQ ID NO: 38 - DNA, 313 bp, Staphylococcus epidermidis
  misc_feature (6)..(6): n is a, c, g, or t
acttcncccnaatcatttgtcccaccttcgacggctagctccaaatggttactccaccggcttcgggtgttacaaactctcgtggtgtgacgggcggtgtgtacaagacccgggaacgtattcaccgtagcatgctgatctacgattactagcgattccagcttcatatagtcgagttgcagactacaatccgaactgagaacaactttatgggatttgcttgacctcgcggtttcgctgccctttgtattgtccattgtagcacgtgtgtagcccaaatcataaggggcatgatgatttgacgtcatcccca

SEQ ID NO: 39 - DNA, 760 bp, Streptococcus salivarius
  misc_feature (9)..(9): n is a, c, g, or t
ggtaatcantactccggctgngtatgaatttnnatttagaaaggaaaaggctataaagaatcataaatcattaataggtgttggaattgtcgctgacgattacccnttgtattttgatgctattaatgaggatggactaggaatggcaggattgaattttcctggaaatgcatattatagcgattttttagagaatgacaaagataatattacgccatttgagtttattccatggattctgggacagtgtagcgatgttaatgaagcaagaaatttagttgaaagaataaatctcattaatcttagttttagcgaacaattacctttagcagggttacattggttaattgcagatagagaaaaatctattgtagtagaagtaactaaatctggcgtacatatttatgataatccaattggagtattgactaataatccggaatttaattatcagatgtataatctgaataaatatcgcaacttatctatcagtacaccacaaaatacattctcagatagcgtggatttaaaggtagacggtactggttttggcggtattggcttaccaggcgatgcatctcccgaatctcgttttgtgagagctgcttttagcaagttaaattcaagtaaagggacgaccgtagaagaagatattactcagtttttccatatactagggacagtannacanataaagggcgttaataagacagaatcaggaaaagaanaatatactgtatattcnaaattgctatgattt

SEQ ID NO: 40 - DNA, 307 bp, Streptococcus salivarius
  misc_feature (12)..(12): n is a, c, g, or t
tcatctatcccnccttaggcggctggctccaaaaggttacctcaccgacttcgggtgttacaaactctcgtggtgtgacgggcggtgtgtacaaggcccgggaacgtattcaccgcggcgtgctgatccgcgattactagcgattccgacttcatgtaggcgagttgcagcctacaatccgaactgagattggctttaagagattagcttgccgtcaccgactcgcaactcgttgtaccaaccattgtagcacgtgtgtagcccaggtcataaggggcatgatgatttgacgtcatccccaccttcc

SEQ ID NO: 41 - DNA, 40 bp, Artificial Sequence
  BSH1 forward primer
atgtgtacagcaattactttaaatggtaatagtaattatt

SEQ ID NO: 42 - DNA, 40 bp, Artificial Sequence
  BSH1 reverse primer
ttaattcaacttatttattacttgttttctttcaaatgga

SEQ ID NO: 43 - DNA, 39 bp, Artificial Sequence
  BSH1 forward primer
atccaattggagtattgactaataatccggaatttaatt

SEQ ID NO: 44 - DNA, 40 bp, Artificial Sequence; identical to SEQ ID NO: 42
  BSH1 reverse primer

SEQ ID NO: 45 - DNA, 19 bp, Artificial Sequence
  16S rRNA forward primer
aggaggtgatccaaccgca

SEQ ID NO: 46 - DNA, 20 bp, Artificial Sequence
  16S rRNA reverse primer
aactggaggaaggtggggat